(19) United States
(12) Patent Application Publication
(10) Pub. No.: US 2022/0387405 A1
(43) Pub. Date: Dec. 8, 2022

(54) METHODS OF TREATMENT WITH N-((R)-1-(3-CHLOROPYRIDIN-2-YL)-2,2,2-TRIFLUOROETHYL)-2-((S)-2,6-DIOXOPIPERIDIN-3-YL)-1-OXOISOINDOLINE-5-CARBOXAMIDE

(71) Applicant: CELGENE CORPORATION, Summit, NJ (US)

(72) Inventor: Michael POURDEHNAD, San Francisco, CA (US)

(73) Assignee: CELGENE CORPORATION, Summit, NJ (US)

(21) Appl. No.: 17/737,508

(22) Filed: May 5, 2022

Related U.S. Application Data
(60) Provisional application No. 63/185,285, filed on May 6, 2021.

(51) Int. Cl. A61K 31/444; A61P 35/02

(52) U.S. Cl. CPC A61K 31/444; A61P 35/02

(57) ABSTRACT

Provided herein are methods of treating, preventing, managing, and/or ameliorating acute myeloid leukemia or myelodysplastic syndrome comprising administering N-((R)-1-(3-chloropyridin-2-yl)-2,2,2-trifluoroethyl)-2-((S)-2,6-dioxopiperidin-3-yl)-1-oxoisoindoline-5-carboxamide or a stereoisomer or mixture of stereoisomers, an isotopologue, pharmaceutically acceptable salt, tautomer, solvate, hydrate, co-crystal, clathrate, or polymorph thereof to a patient.

### 1. RELATED APPLICATIONS

This application claims the benefit of U.S. Provisional Application No. 63/185,285, filed May 6, 2021, the disclosure of which is incorporated by reference in its entirety.

### 2. FIELD

Provided herein are methods of treating, preventing, managing, and/or ameliorating acute myeloid leukemia (AML) and/or myelodysplastic syndrome (MDS) with N-((R)-1-(3-chloropyridin-2-yl)-2,2,2-trifluoroethyl)-2-((S)-2,6-dioxopiperidin-3-yl)-1-oxoisoindoline-5-carboxamide or a stereoisomer or a mixture of stereoisomers, an isotopologue, pharmaceutically acceptable salt, tautomer, solvate, hydrate, co-crystal, clathrate, or polymorph thereof. Further provided is a compound for use in methods of treating, preventing, managing, and/or ameliorating AML or MDS, wherein the compound is N-((R)-1-(3-chloropyridin-2-yl)-2,2,2-trifluoroethyl)-2-(S)-2,6-dioxopiperidin-3-yl)-1-oxoisoindoline-5-carboxamide or a stereoisomer or a mixture of stereoisomers, an isotopologue, pharmaceutically acceptable salt, tautomer, solvate, hydrate, co-crystal, clathrate, or polymorph thereof.

### 3. BACKGROUND

Acute myeloid leukemia (AML) is the most reported type of acute leukemia in adults in the United States of America (USA). Based on Surveillance, Epidemiology, and End Results estimates, approximately 19,940 people will be diagnosed with AML in 2020 in the US and 11,180 will die from the disease. For the time span from 2010 to 2016 the estimated relative 5-year survival for people with AML was 28.7%, and between 2013 and 2017 the median age at diagnosis of AML was 68 years.

Acute myeloid leukemia can arise de novo, be secondary to previous cytotoxic chemotherapy, or arise through transformation of existing myelodysplasia. Therapy-related AML arising from exposure to environmental toxins, cytotoxic drugs, or radiation currently accounts for about 5% to 10% of all cases of AML (Leone et al, Haematologica 1999;84(10):937-945). It is estimated that 35% to 40% of patients with myelodysplastic syndromes will go on to develop AML, with the disease often refractory to current therapy (Silverman et al, Cancer Medicine. 5th ed. Hamilton, Canada: B C Decker; 2000. p. 1931-1946). Preexisting myelodysplastic or myeloproliferative disorders are common in older patients with AML, occurring in 24% to 40% of cases (Gajewski et al, J Clin Oncol 1989;7:1637-1645). Patients with secondary AML due to prior hematologic disease have a lesser response to therapy than those with de novo disease.

Myelodysplastic syndromes are an umbrella term for a heterogeneous collection of hematopoietic stem cell disorders primarily affecting older adults. It is estimated that between 2 and 4 cases per 100,000 persons per year are diagnosed with MDS. The elderly is particularly vulnerable with annual incidence rates between 15 and 50 cases per 100,000 persons per year, Steensma et al., The myelodysplastic syndrome(s): a perspective and review highlighting current controversies, Leuk Res 2003;27(2):95-120. The prognosis depends on the individual's risk factors, with a median survival ranging from 5.3 years in low-risk patients to 1.6 and 0.8 years in high- or very high-risk patients, Greenber et al., Revised international prognostic scoring system for myelodysplastic syndromes, Blood. 2012;120(12):2454-65.

There is a continuing need for effective treatments for AML and MDS.

Anti-leukemic activity of N-((R)-1-(3-chloropyridin-2-yl)-2,2,2-trifluoroethyl)-2-((S)-2,6-dioxopiperidin-3-yl)-1-oxoisoindoline-5-carboxamide has been reported in US Application Publication No. US 2020/0377512. There is a need for safe and efficacious dosages and dosing regimens for administration of N-((R)-1-(3-chloropyridin-2-yl)-2,2,2-trifluoroethyl)-2-(S)-2,6-dioxopiperidin-3-yl)-1-oxoisoindoline-5-carboxamide or a stereoisomer or mixture of stereoisomers, an isotopologue, pharmaceutically acceptable salt, tautomer, solvate, hydrate, co-crystal, clathrate, or polymorph thereof for treatment of acute myeloid leukemia (AML) and myelodysplastic syndrome (MDS).

### 4. BRIEF SUMMARY

In one embodiment, provided herein are methods of treating, preventing, managing, and/or ameliorating AML and/or MDS, by administering to a subject N-((R)-1-(3-chloropyridin-2-yl)-2,2,2-trifluoroethyl)-2-((S)-2,6-dioxopiperidin-3-yl)-1- xoisoindoline-5-carboxamide or a stereoisomer or mixture of stereoisomers, an isotopologue, pharmaceutically acceptable salt, tautomer, solvate, hydrate, co-crystal, clathrate, or polymorph thereof (“Compound 1”). In one embodiment, the AML is relapsed or refractory AML. In one embodiment, provided herein is a method of treating of AML by administering to a subject a N-((R)-1-(3-chloropyridin-2-yl)-2,2,2-trifluoroethyl)-2-(S)-2,6-dioxopiperidin-3-yl)-1-oxoisoindoline-5-carboxamide.

In one embodiment, provided herein are methods of treating, preventing, managing, and/or ameliorating a myelodysplastic syndrome (MDS) by administering to a subject a N-((R)-1-(3-chloropyridin-2-yl)-2,2,2-trifluoroethyl)-2-((S)-2,6-dioxopiperidin-3-yl)-1-oxoisoindoline-5-carboxamide or a stereoisomer or mixture of stereoisomers, an isotopologue, pharmaceutically acceptable salt, tautomer, solvate, hydrate, co-crystal, clathrate, or polymorph thereof (“Compound 1”). In one embodiment, the MDS is relapsed, resistant or refractory MDS. In one embodiment, the MDS is relapsed or refractory MDS. In one embodiment, the MDS is relapsed or refractory higher-risk MDS. In one embodiment, provided herein is a method of treating of MDS by administering to a subject a N-((R)-1-(3-chloropyridin-2-yl)-2,2,2-trifluoroethyl)-2-((S)-2,6-dioxopiperidin-3-yl)-1-oxoisoindoline-5-carboxamide.

In one embodiment, provided herein are methods of treating, preventing, managing, and/or ameliorating AML and/or MDS by administering to a subject an effective amount of Compound 1 in a cycle, wherein the cycle comprises administering Compound 1 in a dose of about 0.1 mg to about 20 mg on days 1 to 7 of a 28 day cycle, days 1 to 5 of a 28 day cycle, days 1 to 10 of a 28 day cycle or days 1 to 14 of a 28 day cycle. In one embodiment, the cycle comprises administering Compound 1 in a dose of about 0.1 mg to about 20 mg on days 1, 4, 8, and 11 per 28-day cycle; days 1, 4, 8, 11, 15, and 18 per 28-day cycle or days 1, 4, 8, 11, 15, 18, 22, and 25 per 28-day cycle.

In one embodiment, provided herein are methods of treating, preventing, managing, and/or ameliorating AML and/or MDS by administering to a subject an effective amount of Compound 1 in a 28 day cycle, wherein the cycle comprises administering Compound 1 in a dose of about 0.1 mg to about 20 mg daily for days 1-7 followed by a 21-day recovery period. In one embodiment, provided herein are methods of treating, preventing, managing, and/or ameliorating AML and/or MDS by administering to a subject an effective amount of Compound 1 in a 28 day cycle, wherein the cycle comprises administering Compound 1 in a dose of about 0.1 mg to about 20 mg daily for days 1-5. In one embodiment, provided herein are methods of treating, preventing, managing, and/or ameliorating AML and/or MDS by administering to a subject an effective amount of Compound 1 in a 28 day cycle, wherein the cycle comprises administering Compound 1 in a dose of about 0.1 mg to about 20 mg daily for days 1-10. In one embodiment, provided herein are methods of treating, preventing, managing, and/or ameliorating AML and/or MDS by administering to a subject an effective amount of Compound 1 in a 28 day cycle, wherein the cycle comprises administering Compound 1 in a dose of about 0.1 mg to about 20 mg daily for days 1-14.

In one embodiment, provided herein are methods of treating, preventing, managing, and/or ameliorating AML and/or MDS by administering to a subject an effective amount of Compound 1 in a cycle, wherein the cycle comprises administering Compound 1 in a dose of about 0.1 mg to about 20 mg twice a week dosing schedule. In one embodiment, provided herein are methods of treating, preventing, managing, and/or ameliorating AML and/or MDS by administering to a subject an effective amount of Compound 1 in a cycle, wherein the cycle comprises administering Compound 1 in a dose of about 0.1 mg to about 20 mg twice a week dosing schedule for 2 weeks, i.e., administering on days 1, 4, 8 and 11 per 28 day cycle, or for 3 weeks, i.e., administering on days 1, 4, 8, 11, 15 and 18 per 28 day cycle. In one embodiment, provided herein are methods of treating, preventing, managing, and/or ameliorating AML and/or MDS by administering to a subject an effective amount of Compound 1 in a cycle, wherein the cycle comprises administering Compound 1 in a dose of about 0.1 mg to about 20 mg on days 1, 4, 8, 11, 15, 18, 22 and 25 per 28 day cycle.

In certain embodiments, provided herein are pharmaceutical compositions, single unit dosage forms, and kits comprising Compound 1 suitable for use in treating, preventing, ameliorating and/or managing AML, and more particularly relapsed or refractory AML. In certain embodiments, provided herein are pharmaceutical compositions, single unit dosage forms, and kits comprising Compound 1 suitable for use in treating, preventing, ameliorating and/or managing MDS, and more particularly relapsed or refractory MDS or relapsed or refractory higher risk MDS. In certain embodiments, such compositions include Compound 1 optionally in combination with one or more other therapeutic agents. In certain embodiments, provided herein are pharmaceutical compositions, comprising Compound 1 for use in treating AML, and more particularly relapsed or refractory AML. In certain embodiments, provided herein are pharmaceutical compositions comprising Compound 1 for use in treating MDS, and more particularly relapsed or refractory MDS or relapsed or refractory higher risk MDS. In certain embodiments, such compositions include Compound 1 optionally in combination with one or more other therapeutic agents.

These and other aspects of the subject matter described herein will become evident upon reference to the following detailed description.

### 5. BRIEF DESCRIPTION OF DRAWINGS

FIG. 1 provides a study design for dose escalation in a daily dosing schedule for Compound 1 on days 1-7.

### 6. DETAILED DESCRIPTION

### 6.1 Definitions

Unless defined otherwise, all technical and scientific terms used herein have the same meaning as is commonly understood by one of ordinary skill in the art. All patents, applications, published applications and other publications are incorporated by reference in their entirety. In the event that there is a plurality of definitions for a term herein, those in this section prevail unless stated otherwise.

The term Compound 1 refers to “N-((R)-1-(3-chloropyridin-2-yl)-2,2,2-trifluoroethyl)-2-((S)-2,6-dioxopiperidin-3-yl)-1-oxoisoindoline-5-carboxamide” having the structure:

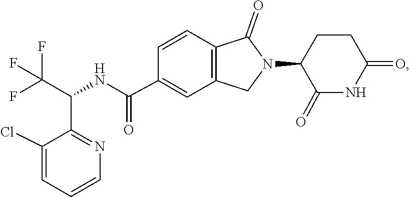 [SMILES: O=C1CC[C@H](N2Cc3cc(C(=O)N[C@H](c4ncccc4Cl)C(F)(F)F)ccc3C2=O)C(=O)N1]

and its stereoisomers or mixture of stereoisomers, isotopologues, pharmaceutically acceptable salts, tautomers, solvates, hydrates, co-crystals, clathrates, or polymorphs thereof. In certain embodiments, Compound 1 refers to N-((R)-1-(3-chloropyridin-2-yl)-2,2,2-trifluoroethyl)-2-((S)-2,6-dioxopiperidin-3-yl)-1-oxoisoindoline-5-carboxamide and its tautomers.

The term “subject” or “patient” refers to an animal, including, but not limited to, a mammal, including a primate (e.g., human), cow, sheep, goat, horse, dog, cat, rabbit, rat, or mouse. The terms “subject” and “patient” are used interchangeably herein in reference, for example, to a mammalian subject, such as a human subject.

In one embodiment, the subject has acute myelogenous or myeloid leukemia (AML), including, for example, the following subtypes of AML. The term “acute myelogenous or myeloid leukemia” refers to hematological conditions characterized by proliferation and accumulation of primarily undifferentiated or minimally differentiated myeloid cells in the bone marrow, and includes subtypes categorized by either the FAB (French, American, British) or WHO classification system. As described herein, the AML includes the following subtypes based on the FAB classification: M0 (AML minimally differentiated); M1 (AML with minimal maturation); M2 (AML with maturation); M3 (Acute promyelocytic leukemia); M4 (Acute myelomonocytic leukemia); M4 (eos Acute myelomonocytic leukemia with eosinophilia); M5 (Acute monocytic leukemia); M6 (Acute erythroid leukemia); and M7 (Acute megakaryoblastic leukemia). As described herein, the AML includes the following subtypes based on the WHO classification: AML with recurrent genetic abnormalities (AML with translocation between chromosomes 8 and 21; AML with translocation or inversion in chromosome 16; AML with translocation between chromosomes 9 and 11; APL (M3) with translocation between chromosomes 15 and 17; AML with translocation between chromosomes 6 and 9; AML with translocation or inversion in chromosome 3); AML (megakaryoblastic) with a translocation between chromosomes 1 and 22; AML with myelodysplasia-related changes; AML related to previous chemotherapy or radiation (Alkylating agent-related AML; Topoisomerase II inhibitor-related AML); AML not otherwise categorized (AML that does not fall into the above categories, i.e. AML minimally differentiated (MO); AML with minimal maturation (M1); AML with maturation (M2); Acute myelomonocytic leukemia (M4); Acute monocytic leukemia (M5); Acute erythroid leukemia (M6); Acute megakaryoblastic leukemia (M7); Acute basophilic leukemia; Acute panmyelosis with fibrosis); Myeloid Sarcoma (also known as granulocytic sarcoma, chloroma or extramedullary myeloblastoma); and Undifferentiated and biphenotypic acute leukemias (also known as mixed phenotype acute leukemias). (see https://www.cancer.org/cancer/acute-myeloid-leukemia/detection-diagnosis-staging/how-classified.html, last accessed May 25, 2017).

In certain embodiments, the risk groups for AML based on cytogenetics are as described below:

| Risk Status | Cytogenetics | Molecular Abnormalities^{a} |
|---|---|---|
| Favorable-risk | Core binding factor: inv(16)^{b,c,d}or | Normal cytogenetics: |
| | t(16; 16)^{b,c,d} or t(8; 21)^{b,d} or | NPM1 mutation in the absence of |
| | t(15; 17)^{d} | FLT3-ITD or isolated biallelic |
| | | CEBPA mutation |
| Intermediate-risk | Normal cytogenetics | Core binding factor with |
| | +8 alone | c-KIT mutation^{b} |
| | t(9; 11) | |
| | Other non-defined | |
| Poor-risk | Complex (≥3 clonal | Normal cytogenetics: |
| | chromosomal abnormalities) | with FLT3-ITD mutation ^{f} |
| | Monosomal karyotype | TP53 mutation |
| | -5, 5q-, -7, 7q- | |
| | 11q23-non t(9; 11) | |
| | inv(3), t(3; 3) | |
| | t(6; 9) | |
| | t(9; 22)^{e} | |

^{a}The molecular abnormalities included in this table reflect those for which validated assays are available in standardized commercial laboratories.
^{b}Emerging data indicate that the presence of KIT mutations in patients with t(8; 21), and to a lesser extent inv (16), confers a higher risk of relapse. These patients are considered intermediate risk and should be considered for hematopoietic stem cell transplant (HSCT) or clinical trials, if available. Other cytogenetic abnormalities in addition to these finding do not alter risk status.
^{c}Paschka P, et al. Blood 2013;121:170-177.
^{d}Other cytogenetic abnormalities in addition to these findings do not alter better risk status
^{e}For Philadelphia + acute myeloid leukemia (AML) t(9; 22), manage as myeloid blast crisis in chronic myeloid leukemia (CML), with addition of tyrosine kinase inhibitors.

In one embodiment, the subject has myelodysplastic syndrome (MDS), including, for example, the following subtypes of MDS. The term “myelodysplastic syndrome” refers to hematological conditions characterized by abnormalities in the production of one or more of the cellular components of blood (red cells, white cells (other than lymphocytes) and platelets (or their progenitor cells, megakaryocytes)), and includes the following disorders: refractory anemia (RA); RA with ringed sideroblasts (RARS); RA with excess of blasts (RAEB); refractory cytopenia with multilineage dysplasia (RCMD), refractory cytopenia with unilineage dysplasia (RCUD); unclassifiable myelodysplastic syndrome (MDS-U), myelodysplastic syndrome associated with an isolated del(5q) chromosome abnormality, therapy-related myeloid neoplasms and chronic myelomonocytic leukemia (CMML). The MDS as used herein also includes very low risk, low risk, intermediate risk, high risk and very high risk MDS. In one embodiment, the MDS is primary or de novo MDS. In other embodiments, the MDS is secondary. In one embodiment, the MDS is relapsed or refractory MDS. In one embodiment, the MDS is relapsed or refractory higher risk MDS.

In certain embodiments, MDS is classified based on the World Health Organization (WHO) classification of MDS as described below:

WHO classifications for MDS

| WHO myeloid neoplasm and | Dysplastic | | PB and BM findings and |
|---|---|---|---|
| acute leukemia classification | findings | Cytopenias^{a} | cytogenetics |
| MDS with single lineage | 1 | 1 or 2 | BM <5%, PB <1%, |
| dysplasia (MDS-SLD) | | | no Auer Rods |
| | | | Any cytogenetics, unless |
| | | | fulfills all criteria for MDS |
| | | | with isolated del(5q) |
| MDS with ring sideroblasts | 1 | 1 or 2 | BM <5%, PB <1%, |
| (MDS-RS)^{b} | 2 or 3 | 3 | no Auer Rods |
| MDS-RS and single lineage | | | Any cytogenetics, unless |
| dysplasia | | | fulfills all criteria for MDS |
| MDS-RS and multilineage | | | with isolated del(5q) |
| dysplasia | | | |
| MDS with multilineage | 2 or 3 | 1-3 | BM <5%, PB <1%, |
| dysplasia (MDS-MLD) | | | no Auer Rods |
| | | | Any cytogenetics, unless |
| | | | fulfills all criteria for MDS |
| | | | with isolated del(5q) |
| MDS with excess | | | |
| blasts (MDS-EB) | | | |
| MDS-EB-1 | 0-3 | 1-3 | BM 5-9% or PB 2-4%, |
| | | | no Auer Rods |
| | | | Any cytogenetics |
| MDS-EB-2 | 0-3 | 1-3 | BM 10-19% or PB 5-19% |
| | | | or Auer Rods |
| | | | Any cytogenetics |
| MDS with isolated del(5q) | 1-3 | 1-2 | BM <5%, PB <1%, |
| | | | no Auer Rods |
| | | | del(5q) alone or with 1 |
| | | | additional abnormality |
| | | | except −7 or del(7q) |
| MDS, | | | |
| unclassifiable (MDS-U) | | | |
| MDS-U with 1% blood blasts | 1-3 | 1-3 | BM <5%, PB = 1%^{c}, |
| | | | no Auer Rods |
| | | | Any cytogenetics |
| MDS-U with SLD and | 1 | 3 | BM <5%, PB <1%, |
| pancytopenia | | | no Auer Rods |
| | | | Any cytogenetics |
| MDS-U based on defining | 0 | 1-3 | BM <5%, PB <1%, |
| cytogenetic abnormality | | | no Auer Rods |
| | | | MDS-defining abnormality^{d} |

^{a}Cytopenias defined as: hemoglobin, <10 g/dL, p atelet count, <100 × 10⁹/L; and absolute neutrophil count, <1.8 × 10⁹/L. Rarely, MDS may present with mild anemia or thrombocytopenia above these levels. Peripheral blood monocytes must be <1 × 10⁹/L.
^{b}Cases with ≥15% ring sideroblasts by definition have significant erythroid dysplasia, and are classified as MDS-RS-SLD.
^{c}One percent PB blasts must be recorded on at least 2 separate occasions.
^{d}Abnormality must be demonstrated by conventional karyotyping, not by FISH or sequencing.

The presence of +8, −Y, of del(20q) is not considered to be MDS-defining in the absence of diagnostic morphologic features of MDS. Arber, et al. Blood 2016;127(20):2391-2405, and Vardiman, et al. Blood. 2009; 114(5):937-51.

As used herein, and unless otherwise specified, the terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more prophylactic or therapeutic agents to a patient with such a disease or disorder. In some embodiments, the terms refer to the administration of a compound provided herein, with or without other additional active agent, after the onset of symptoms of the particular disease. In one embodiment, the disease is AML, including, a subtype of AML discussed above. In one embodiment, the disease is MDS, including, a subtype of MDS discussed above.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound provided herein, with or without other additional active compound, prior to the onset of symptoms, particularly to patients at risk of diseases or disorders provided herein. The terms encompass the inhibition or reduction of a symptom of the particular disease. Patients with familial history of a disease in particular are candidates for preventive regimens in certain embodiments. In addition, patients who have a history of recurring symptoms are also potential candidates for the prevention. In this regard, the term “prevention” may be interchangeably used with the term “prophylactic treatment.” In one embodiment, the disease is AML, including, a subtype of AML discussed herein. In one embodiment, the disease is MDS, including, a subtype of MDS discussed herein.

As used herein, and unless otherwise specified, the terms “manage,” “managing” and “management” refer to preventing or slowing the progression, spread or worsening of a disease or disorder, or of one or more symptoms thereof. Often, the beneficial effects that a patient derives from a prophylactic and/or therapeutic agent do not result in a cure of the disease or disorder. In this regard, the term “managing” encompasses treating a patient who had suffered from the particular disease in an attempt to prevent or minimize the recurrence of the disease, or lengthening the time during which the remains in remission. In one embodiment, the disease is AML, including, a subtype of AML discussed herein. In one embodiment, the disease is MDS, including a subtype of MDS discussed herein.

The term “adverse effect” is used according to its ordinary and common meaning in the art and as used herein can refer to a specific condition associated with treatment, prevention, management, or amelioration of a disease described herein resulting from treatment with a compound or composition described herein. One such adverse effect is the onset of neutropenia. Neutropenia can result from damage to bone marrow, and refers to any condition causing inhibition, elimination, or disruption (directly or indirectly) of neutrophil production and/or maturation.

The term “refractory or resistant” refers to a circumstance where a subject or a mammal, even after intensive treatment, has residual cancer cells in his body.

The term “drug resistance” refers to the condition when a disease does not respond to the treatment of a certain drug or drugs. Drug resistance can be either intrinsic, which means the disease has never been responsive to the particular drug or drugs, or it can be acquired, which means the disease ceases responding to particular a drug or drugs that the disease had previously responded to. In certain embodiments, drug resistance is intrinsic. In certain embodiments, the drug resistance is acquired.

The term “relapsed” refers to a situation where a subject or a mammal, which has had a remission of cancer after therapy has a return of cancer cells.

A “cycling therapy” refers to a regimen or therapy that includes an administration period as described herein and a rest period as described herein.

The term “administration period” as used herein refers to a period of time a subject is continuously or actively administered a compound or composition described herein.

The term “rest period” as used herein refers to a period of time, often following an administration period, where a subject is not administered a compound or composition described herein (e.g. discontinuation of treatment). In certain embodiments, a “rest period” refers to a period of time where a single agent is not administered to a subject or treatment using a particular compound is discontinued. In such embodiments, a second therapeutic agent (e.g., a different agent than the compound or composition administered in the previous administration period) can be administered to the subject.

The term “QD” refers to a once daily dose administration.

The terms “determining”, “measuring”, “evaluating”, “assessing” and “assaying” as used herein generally refer to any form of measurement, and include determining if an element is present or not. These terms include both quantitative and/or qualitative determinations. Assessing may be relative or absolute. “Assessing the presence of” can include determining the amount of something present, as well as determining whether it is present or absent.

As used herein, and unless otherwise specified, a “therapeutically effective amount” of a compound is an amount sufficient to provide a therapeutic benefit in the treatment or management of a disease or disorder, or to delay or minimize one or more symptoms associated with the disease or disorder. A therapeutically effective amount of a compound means an amount of therapeutic agent, alone or in combination with other therapies, which provides a therapeutic benefit in the treatment or management of the disease or disorder. The term “therapeutically effective amount” can encompass an amount that improves overall therapy, reduces or avoids symptoms or causes of disease or disorder, or enhances the therapeutic efficacy of another therapeutic agent.

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

As used herein, ECOG status refers to Eastern Cooperative Oncology Group (ECOG) Performance Status (Oken M, et al Toxicity and response criteria of the Eastern Cooperative Oncology Group. Am J Clin Oncol 1982;5(6):649-655), as shown below:

| Score | Description |
|---|---|
| 0 | Fully active, able to carry on all pre-disease performance without restriction |
| 1 | Restricted in physically strenuous activity but ambulatory and able to carry out |
| | work of a light or sedentary nature, eg, light housework, office work. |
| 2 | Ambulatory and capable of all self-care but unable to carry out any work |
| | activities. Up and about more than 50% of waking hours. |
| 3 | Capable of only limited self-care, confined to bed or chair more than 50% of |
| | waking hours. |
| 4 | Completely disabled. Cannot carry on any self-care. Totally confined to bed |
| | or chair |
| 5 | Dead |

As used herein, Overall survival (OS) means the time from randomization in a clinical trial until death from any cause. Progression-free survival (PFS) means the time from randomization in a clinical trial until progression or death. Event-free survival (EFS) means the time from study entry until any treatment failure, including disease progression, treatment discontinuation for any reason, or death. Overall response rate (ORR) means the sum of the percentage of patients who achieve complete and partial responses. Duration of response (DoR) is the time from achieving a response until relapse or disease progression.

As used herein, “patient population treated with Compound 1” refers to a patient population that has received any treatment with Compound 1.

As used herein, “patient population not treated with Compound 1” refers to a patient population that has not received any treatment with Compound 1. Such patient population includes patients who have not received any treatment for cancer, patients who have been treated with placebo, and patients who have been treated with any cancer therapy, other than treatment with Compound 1.

In AML patients, response to treatment can be assessed based on the International Working Group Response Criteria in AML (Cheson et al. Revised recommendations of the International Working Group for diagnosis, standardization of response criteria, treatment outcomes, and reporting standards for therapeutic trials in acute myeloid leukemia. J Clin Oncol 2003; 21(24):4642-9.

| | | | | Bone | |
|---|---|---|---|---|---|
| | | | | Marrow | |
| Response | Time of | Neutrophils | Platelets | Blasts | |
| Criterion | Assessment | (μL) | (μL) | (%) | Other |
| Early Treatment | 7-10 days | NA | NA | <5 | |
| assessment | after therapy | | | | |
| Morphologic | Varies by | NA | NA | <5 | Flow |
| Leukemia-free | protocol | | | | cytometry EMD |
| State | | | | | |
| Morphologic | Varies by | ≥1,000 | ≥100,000 | <5 | Transfusion |
| CR | protocol | | | | EMD |
| Cytogenetic | Varies by | ≥1,000 | ≥100,000 | <5 | Cytogenetics- |
| CR (CRc) | protocol | | | | normal, EMD |
| Molecular | Varies by | ≥1,000 | ≥100,000 | <5 | Molecular- |
| CR (CRm) | protocol | | | | negative, EMD |
| Morphologic | Varies by | Fulfill all criteria for CR except | | | |
| CR with | protocol | for residual neutropenia (<1,000/μL) | | | |
| incomplete | | or thrombocytopenia (<100,000/μL). | | | |
| blood recovery | | | | | |
| (CRi) | | | | | |
| Partial | Varies by | ≥1,000 | ≥100,000 | Decrease | Blasts ≤5% if |
| Remission | protocol | | | ≥50 | Auer rod positive |
| | | | | resulting | |
| | | | | in 5 to 25 | |

| | | |
|---|---|---|
| Relapse | Varies by | Reappearance of leukemic blasts in the |
| after CR | protocol | peripheral blood or ≥5% blasts in the bone marrow not |
| | | attributable to any other cause (eg, bone marrow |
| | | regeneration after consolidation therapy). |

Key: AML = acute myelogenous leukemia;
CR = complete remission;
EMD = extramedullary disease;
IWG = International Working Group;
NA = not applicable.

The treatment of MDS may be assessed by International Working Group (IWG) Response Criteria for Myelodysplasia.

Modified IWG Response Criteria for MDS

| Category | Response criteria (responses must last at least 4 weeks) |
|---|---|
| Complete remission (CR) | Bone marrow: ≤5% myeloblasts with normal maturation of all |
| | cell lines^{a} |
| | Persistent dysplasia will be noted^{a,b} |
| | Peripheral blood^{c} |
| | Hemoglobin ≥11 g/dL |
| | Platelets ≥100 × 10⁹/L |
| | Neutrophils ≥1.0 × 10⁹/L^{b} |
| | Blasts 0% |
| Partial remission (PR) | All CR criteria if abnormal before treatment, except: |
| | Bone marrow blasts decreased by ≥50% over |
| | pretreatment but still >5% |
| | Cellularity and morphology not relevant |
| Marrow CR^{b} ± | Bone marrow: ≤5% myeloblasts and decrease by ≥50% over |
| Hematologic | pretreatment^{b} Note: Blasts at baseline must be ≥5% in order for |
| Improvement (HI) | subject to be evaluable for Marrow CR^{d} |
| | Peripheral blood: if HI responses, they will be noted in addition to |
| | marrow CR^{b} |
| Stable disease (SD) | Failure to achieve at least PR, but no evidence of progression for |
| | >8 weeks |
| Failure | Death during treatment or disease progression characterized by |
| | worsening of cytopenias, increase in percentage of bone marrow |
| | blasts, or progression to a more advanced MDS FAB subtype |
| | than pretreatment |
| Relapse after CR or PR | At least 1 of the following: |
| | Return to pretreatment bone marrow blast percentage |
| | Decrement of ≥50% from maximum remission/response |
| | levels in granulocytes or platelets |
| | Reduction of Hgb concentration by ≥1.5 g/dL or transfusion |
| | dependence |
| Cytogenetic Response | Complete-Disappearance of the chromosomal abnormality |
| | without appearance of new ones |
| | Partial-At least 50% reduction of the chromosomal abnormality |
| Disease Progression (PD) | For patients with: |
| | Less than 5% blasts: ≥50% increase in blasts to >5% blasts |
| | 5%-10% blasts: ≥50% increase in blasts to >10% blasts |
| | 10%-20% blasts: ≥50% increase in blasts to >20% blasts |
| | Any of the following: |
| | At least 50% decrement from maximum remission/response |
| | levels in granulocytes or platelets |
| | Reduction in Hgb concentration by ≥2 g/dL |
| | Transfusion dependence |
| Disease transformation | Transformation to AML (20% or more BM or PB blasts)^{d} |
| Hematologic | |
| Improvement (HI) | |
| Erythroid response | Hgb increase by ≥1.5 g/dL |
| (HI-E) | Relevant reduction of units of RBC transfusions by an absolute |
| (Pretreatment <11 g/dL) | number of at least 4 RBC transfusions/8 weeks compared with the |
| | pretreatment transfusion number in the previous 8 weeks. Only |
| | RBC transfusions given for a Hgb of ≤9.0 g/dL pretreatment will |
| | count in the RBC transfusion evaluation |
| Platelet response (HI-P) | Absolute increase of ≥30 × 10⁹/L for patients starting |
| (Pretreatment < | with >20 × 10⁹/L |
| 100 × 10^{9/}L) | Increase from <20 × 10⁹/L to >20 × 10⁹/L and by at least 100% |
| Neutrophil response | At least 100% increase and an absolute increase of >0.5 × 10⁹/L |
| (HI-N) | |
| (Pretreatment < | |
| 1.0 × 10⁹/L) | |
| Progression/relapse | At least one of the following: |
| after HI | At least 50% decrement from maximum response levels in |
| | granulocytes or platelets |
| | Reduction in Hgb by ≥1.5 g/dL |
| | Transfusion dependence |

BM = bone marrow;
CR = complete remission;
FAB = French-American-British;
Hgb = hemoglobin;
HI = hematologic improvement;
IWG = International Working Group;
MDS = myelodysplastic syndromes;
PB = peripheral blood;
PD = Disease Progression;
PR = partial remission;
RBC = red blood cell.
^{a}Dysplastic changes should consider the normal range of dysplastic changes (modification).
^{b}Modification to IWG response criteria.
^{c}In some circumstances, protocol therapy may require the initiation of further treatment (eg, consolidation, maintenance) before the 4-week period. Such subjects can be included in the response category into which they fit at the time the therapy is started. Transient cytopenias during repeated chemotherapy courses should not be considered as interrupting durability of response, as long as they recover to the improved counts of the previous course.
^{d}Sponsor modification of IWG criteria.

### Sources: Cheson, 2006 and Vardiman, 2008.

RBC and Platelet Transfusion Independence

| | At Screening | During Study Treatment |
|---|---|---|
| RBC transfusion | Subjects who received | Subjects who experienced a Hgb increase of |
| independence | <4 RBC units during | 1.5 g/dL over baseline and who received no |
| | the previous 56 days | RBC transfusions during a 56-day period on |
| | | treatment. Note: Only RBC transfusions given |
| | | for a Hgb of ≤9.0 g/dL within 3 days prior to |
| | | the transfusion will count in the RBC |
| | | transfusion response evaluation |
| RBC transfusion | Subjects who received | |
| dependence | ≥4 RBC units during | |
| | the previous 56 days | |
| Platelet | Subjects who received | Subjects who received no platelet transfusions |
| transfusion | <2 platelet transfusions | during a 56-day period on treatment |
| independence | during the previous | |
| | 56 days | |
| Platelet | Subjects who received | |
| transfusion | ≥2 platelet transfusions | |
| dependence | during the previous | |
| | 56 days. | |

RBC = red blood cell;
Hgb = hemoglobin.
^{a} RBC transfusion independence and RBC transfusion dependence are defined according to modified IWG criteria.
^{b} Platelet transfusior independence and platelet transfusion dependence are defined by the Sponsor.

Source: Cheson, et al. Blood. 2006;108(2):419-25.

Revised International Prognostic Scoring System is used for prognosis of MDS as follows:

IPSS-R Cytogenetic Risk Group

| Cytogenetic | |
|---|---|
| Prognostic | |
| Subgroups | Cytogenetic Abnormalities |
| Very good | −Y, del(11q) |
| Good | Normal, del(5q), del(12p), del(20q), |
| | double including del(5q) |
| Intermediate | del(7q), +8, +19, i(l7q), any other |
| | single or double independent clones |
| Poor | −7, inv(3)/t(3q)/del(3q), |
| | double including −7/del(7q), |
| | Complex: 3 abnormalities |
| Very poor | Complex: >3 abnormalities |

Source: Greenburg, et al. Blood. 2012;120(12):2454-65.

IPSS-R Prognostic Score Values

| Prognostic | | | | | | | |
|---|---|---|---|---|---|---|---|
| variable | 0 | 0.5 | 1 | 1.5 | 2 | 3 | 4 |
| Cytogenetics | Very | — | Good | — | Inter- | Poor | Very |
| | Good | | | | mediate | | Poor |
| Bone Marrow | ≤2 | — | >2-<5 | — | 5-10 | >10 | — |
| Blast (%) | | | | | | | |
| Hemoglobin | ≥10 | — | 8-<10 | <8 | — | — | — |
| (g/dL) | | | | | | | |
| Platelets | ≥100 | 50-<100 | <50 | — | — | — | — |
| (×10⁹/L) | | | | | | | |
| ANC (×10⁹/L) | ≥0.8 | <0.8 | — | — | — | — | — |

Source: Greenburg, et al. Blood. 2012;120(12):2454-65.

The total IPSS-R score is calculated as the sum of the cytogenetics, bone marrow blast percentage, hemoglobin, platelets and ANC individual scores.

IPSS-R Prognostic Risk Categories/Scores

| Risk Category | Risk Score |
|---|---|
| Very Low | ≤1.5 |
| Low | >1.5-3 |
| Intermediate | >3-4.5 |
| High | >4.5-6 |
| Very High | >6 |

Source: Greenburg, et al. Blood. 2012;120(12):2454-65.

IPSS-R: Prognostic Risk Category Clinical Outcomes

| Prognostic | No. | Very | | | | Very |
|---|---|---|---|---|---|---|
| variable | pts | Low | Low | Intermediate | High | High |
| Patients, % | 7012 | 19% | 38% | 20% | 13% | 10% |
| Median Overall | — | 8.8 | 5.3 | 3.0 | 1.6 | 0.8 |
| Survival (years) | | | | | | |
| Median time to 25% | — | Not | 10.8 | 3.2 | 1.4 | 0.73 |
| AML evolution | | reached | | | | |

Source: Greenberg, et al. Blood. 2012;120(12):2454-65

As used herein and unless otherwise indicated, the term “pharmaceutically acceptable salt” includes, but is not limited to, a salt of an acidic group. Under certain acidic conditions, the compound can form a wide variety of salts with various inorganic and organic acids. The acids that can be used to prepare pharmaceutically acceptable salts of such basic compounds are those that form salts such as pharmacologically acceptable anions including, but not limited to, acetate, benzenesulfonate, benzoate, bicarbonate, bitartrate, bromide, calcium edetate, camsylate, carbonate, chloride, bromide, iodide, citrate, dihydrochloride, edetate, edisylate, estolate, esylate, fumarate, gluceptate, gluconate, glutamate, glycollylarsanilate, hexylresorcinate, hydrabamine, hydroxynaphthoate, isethionate, lactate, lactobionate, malate, maleate, mandelate, methanesulfonate (mesylate), methylsulfate, muscate, napsylate, nitrate, pantothenate, phosphate/diphosphate, polygalacturonate, salicylate, stearate, succinate, sulfate, tannate, tartrate, teoclate, triethiodide, and pamoate.

As used herein and unless otherwise indicated, the term “hydrate” means a compound provided herein or a salt thereof, further including a stoichiometric or non-stoichiometric amount of water bound by non-covalent intermolecular forces. The hydrates can be crystalline or non-crystalline.

As used herein and unless otherwise indicated, the term “solvate” means a solvate formed from the association of one or more solvent molecules to compound provided herein. The term “solvate” includes hydrates (e.g., monohydrate, dihydrate, trihydrate, tetrahydrate, and the like). The solvates can be crystalline or non-crystalline.

As used herein, and unless otherwise specified, the term “stereoisomer” encompasses all enantiomerically/stereomerically pure and enantiomerically/stereomerically enriched compounds provided herein.

As used herein, and unless otherwise indicated, the term “stereomerically pure” or “enantiomerically pure” means that a compound includes one stereoisomer and is substantially free of its counter stereoisomer or enantiomer. For example, a compound is stereomerically or enantiomerically pure when the compound contains 80%, 90%, or 95% or more of one stereoisomer and 20%, 10%, or 5% or less of the counter stereoisomer. In certain cases, a compound provided herein is considered optically active or stereomerically/enantiomerically pure (i.e., substantially the R-form or substantially the S-form) with respect to a chiral center when the compound is about 80% ee (enantiomeric excess) or greater, preferably, equal to or greater than 90% ee with respect to a particular chiral center, and more preferably 95% ee with respect to a particular chiral center.

As used herein, and unless otherwise indicated, the term “stereomerically enriched” or “enantiomerically enriched” encompasses racemic mixtures as well as other mixtures of stereoisomers of compounds provided herein (e.g., R/S=30/70, 35/65, 40/60, 45/55, 55/45, 60/40, 65/35 and 70/30).

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. 1972, 11:942-944).

### 6.2 Compound

The compound suitable for use in the methods provided herein is Compound 1: N-((R)-1-(3-chloropyridin-2-yl)-2,2,2-trifluoroethyl)-2-((S)-2,6-dioxopiperidin-3-yl)-1-oxoisoindoline-5-carboxamide having the structure:

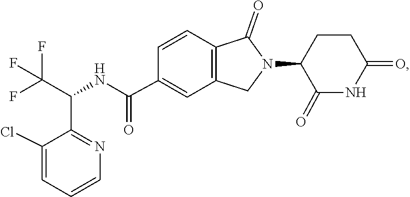 [SMILES: O=C1CC[C@H](N2Cc3cc(C(=O)N[C@H](c4ncccc4Cl)C(F)(F)F)ccc3C2=O)C(=O)N1]

or its stereoisomers or mixture of stereoisomers, isotopologues, pharmaceutically acceptable salts, tautomers, solvates, hydrates, co-crystals, clathrates, or polymorphs thereof. In certain embodiments, Compound 1 refers to N-((R)-1-(3-chloropyridin-2-yl)-2,2,2-trifluoroethyl)-2-((S)-2,6-dioxopiperidin-3-yl)-1-oxoisoindoline-5-carboxamide.

Compound 1 can be prepared according to the methods described in the Examples provided herein or as described in U.S. Patent Publication No. 2020/0377512 A1, the disclosure of which is incorporated herein by reference in its entirety. The compound can be also synthesized according to other methods apparent to those of skill in the art based upon the teaching herein.

Compound 2, N-((R)-1-(3-chloropyridin-2-yl)-2,2,2-trifluoroethyl)-2-((R)-2,6-dioxopiperidin-3-yl)-1-oxoisoindoline-5-carboxamide, has the following structure:

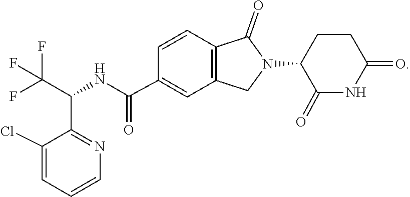 [SMILES: O=C1CC[C@@H](N2Cc3cc(C(=O)N[C@H](c4ncccc4Cl)C(F)(F)F)ccc3C2=O)C(=O)N1]

### 6.3 Methods of Use

In one embodiment, provided herein are methods of treating, preventing, managing, and/or ameliorating AML and/or MDS by administering Compound 1 to a subject.

In one embodiment, provided herein are methods of treating, preventing, managing, and/or ameliorating AML by administering a therapeutically active amount of Compound 1 to a subject. In one embodiment, the AML is relapsed or refractory AML. In some embodiments, the AML is newly diagnosed AML. In some embodiments, the AML is primary AML. In others, the AML is relapsed AML. In still others, the AML is refractory AML. In some embodiments the AML is relapsed/refractory AML. Relapsed or refractory disease may be de novo AML or secondary AML, e.g., therapy-related AML (t-AML). In one embodiment, the AML is refractory to one or more of cytarabine, daunorubicin, idarubicin, midostaurin, cladribine, gemtuzumab ozogamicin, fludarabine, mitoxantrone, gilteritinib, glasdegib, and venetoclax.

In certain embodiments, the methods provided herein encompass the treatment of subjects who have not been previously treated for AML. In some embodiments, the subject has not undergone allogeneic bone marrow transplantation. In some embodiments, the subject has not undergone a stem cell transplantation. In some embodiments, the subject has not received hydroxyurea treatment. In some embodiments, the subject has not been treated with systemic glucocorticoids.

In other embodiments, the methods encompass treating subjects who have been previously treated or are currently being treated for AML. For example, the subject may have been previously treated or is currently being treated with a standard treatment regimen for AML. The subject may have been treated with any standard AML treatment regimen known to the practitioner of skill in the art. In certain embodiments, the subject has been previously treated with at least one induction/reinduction or consolidation AML regimen. In some embodiments, the subject has undergone autologous bone marrow transplantation or stem cell transplantation as part of a consolidation regimen. In some embodiments, the subject has undergone hydroxyurea treatment. In some embodiments, the subject has undergone prior induction or consolidation therapy with cytarabine (Ara-C). In some embodiments, the subject has undergone treatment with systemic glucocorticosteroids. In other embodiments, the methods encompass treating subjects who have been previously treated for AML, but are non-responsive to standard therapies.

In another embodiment, the AML has FAB classification M0/1. In another embodiment, the AML has FAB classification M2. In another embodiment, the AML has FAB classification M3. In another embodiment, the AML has FAB classification M4. In another embodiment, the AML has FAB classification M5. In one embodiment, the AML is AML with at least one recurrent genetic abnormality (for example, AML with translocation between chromosomes 8 and 21; AML with translocation or inversion in chromosome 16; AML with translocation between chromosomes 9 and 11; APL (M3) with translocation between chromosomes 15 and 17; AML with translocation between chromosomes 6 and 9; AML with translocation or inversion in chromosome 3); AML (megakaryoblastic) with a translocation between chromosomes 1 and 22; AML with myelodysplasia-related changes; AML related to previous chemotherapy or radiation (for example, alkylating agent-related AML; or Topoisomerase II inhibitor-related AML); AML not otherwise categorized (for example, AML that does not fall into the above categories, i.e. AML minimally differentiated (M0); AML with minimal maturation (M1); AML with maturation (M2); Acute myelomonocytic leukemia (M4); Acute monocytic leukemia (M5); Acute erythroid leukemia (M6); Acute megakaryoblastic leukemia (M7); Acute basophilic leukemia; or Acute panmyelosis with fibrosis); Myeloid Sarcoma (also known as granulocytic sarcoma, chloroma or extramedullary myeloblastoma); or Undifferentiated and biphenotypic acute leukemias (also known as mixed phenotype acute leukemias).

In one embodiment, provided herein are methods of treating, preventing, managing, and/or ameliorating AML by administering Compound 1 to a subject in a dose of about 0.1 mg to about 20 mg. In one embodiment, provided herein are methods of treating, preventing, managing, and/or ameliorating relapsed or refractory AML by administering Compound 1 to a subject in a dose of about 0.1 mg to about 20 mg. In one embodiment, provided herein are methods of treating AML by administering Compound 1 to a subject in a dose of about 0.1 mg to about 20 mg. In one embodiment, provided herein are methods of treating relapsed or refractory AML by administering Compound 1 to a subject in a dose of about 0.1 mg to about 20 mg. In one embodiment, provided herein are methods of treating, preventing, managing, and/or ameliorating AML by administering Compound 1 to a subject in a cycle, wherein the cycle comprises administering Compound 1 in a dose of about 0.1 mg to about 20 mg on days 1 to 5, 1 to 7, 1 to 10, or 1 to 14, of a 28 day cycle. In one embodiment, provided herein are methods of treating, preventing, managing, and/or ameliorating relapsed or refractory AML by administering Compound 1 to a subject in a cycle, wherein the cycle comprises administering Compound 1 in a dose of about 0.1 mg to about 20 mg on days 1 to 5, 1 to 7, 1 to 10, or 1 to 14, of a 28 day cycle. In one embodiment, provided herein are methods of treating, preventing, managing, and/or ameliorating AML by administering Compound 1 to a subject in a cycle, wherein the cycle comprises administering Compound 1 in a dose of about 0.1 mg to about 20 mg on days 1 to 5, of a 28 day cycle. In one embodiment, provided herein are methods of treating, preventing, managing, and/or ameliorating relapsed or refractory AML by administering Compound 1 to a subject in a cycle, wherein the cycle comprises administering Compound 1 in a dose of about 0.1 mg to about 20 mg on days 1 to 5, of a 28 day cycle. In one embodiment, provided herein are methods of treating, preventing, managing, and/or ameliorating AML by administering Compound 1 to a subject in a cycle, wherein the cycle comprises administering Compound 1 in a dose of about 0.1 mg to about 20 mg on days 1 to 7, of a 28 day cycle. In one embodiment, provided herein are methods of treating, preventing, managing, and/or ameliorating relapsed or refractory AML by administering Compound 1 to a subject in a cycle, wherein the cycle comprises administering Compound 1 in a dose of about 0.1 mg to about 20 mg on days 1 to 7, of a 28 day cycle. In one embodiment, provided herein are methods of treating, preventing, managing, and/or ameliorating AML by administering Compound 1 to a subject in a cycle, wherein the cycle comprises administering Compound 1 in a dose of about 0.1 mg to about 20 mg on days 1 to 10, of a 28 day cycle. In one embodiment, provided herein are methods of treating, preventing, managing, and/or ameliorating relapsed or refractory AML by administering Compound 1 to a subject in a cycle, wherein the cycle comprises administering Compound 1 in a dose of about 0.1 mg to about 20 mg on days 1 to 10, of a 28 day cycle. In one embodiment, provided herein are methods of treating, preventing, managing, and/or ameliorating AML by administering Compound 1 to a subject in a cycle, wherein the cycle comprises administering Compound 1 in a dose of about 0.1 mg to about 20 mg on 1 to 14, of a 28 day cycle. In one embodiment, provided herein are methods of treating, preventing, managing, and/or ameliorating relapsed or refractory AML by administering Compound 1 to a subject in a cycle, wherein the cycle comprises administering Compound 1 in a dose of about 0.1 mg to about 20 mg on 1 to 14, of a 28 day cycle.

In one embodiment, provided herein are methods of treating AML by administering Compound 1 to a subject in a dose of about 0.1 mg to about 20 mg in a cycle, wherein the cycle comprises administering Compound 1 on days 1, 4, 8, 11, 15, 18, 22, 25 of a 28 day cycle. In one embodiment, provided herein are methods of treating relapsed or refractory AML by administering Compound 1 to a subject in a dose of about 0.1 mg to about 20 mg in a cycle, wherein the cycle comprises administering Compound 1 on days 1, 4, 8, 11, 15, 18, 22, 25 of a 28 day cycle. In one embodiment, provided herein are methods of treating AML by administering Compound 1 to a subject in a dose of about 0.1 mg to about 20 mg in a cycle, wherein the cycle comprises administering Compound 1 on days 1, 4, 8, 11, 15, 18 of a 28 day cycle. In one embodiment, provided herein are methods of treating relapsed or refractory AML by administering Compound 1 to a subject in a dose of about 0.1 mg to about 20 mg in a cycle, wherein the cycle comprises administering Compound 1 on days 1, 4, 8, 11, 15, 18 of a 28 day cycle. In one embodiment, provided herein are methods of treating AML by administering Compound 1 to a subject in a dose of about 0.1 mg to about 20 mg in a cycle, wherein the cycle comprises administering Compound 1 on days 1, 4, 8, 11 of a 28 day cycle. In one embodiment, provided herein are methods of treating relapsed or refractory AML by administering Compound 1 to a subject in a dose of about 0.1 mg to about 20 mg in a cycle, wherein the cycle comprises administering Compound 1 on days 1, 4, 8, 11 of a 28 day cycle.

In some such embodiments, provided herein are methods of treating, preventing, managing, and/or ameliorating AML comprising administering Compound 1 a dose of about 0.1 mg/day, 0.2 mg/day, 0.4 mg/day, 0.5 mg/day, 0.8 mg/day, 1 mg/day, 1.5 mg/day, 2 mg/day, 3 mg/day, 6 mg/day, 9 mg/day and 12 mg/day.

In one embodiment, provided herein are methods of treating, preventing, managing, and/or ameliorating a myelodysplastic syndrome (MDS) by administering a therapeutically active amount of Compound 1 to a subject. In one embodiment provided herein is a method of treating MDS. In one embodiment, the MDS is relapsed, resistant or refractory MDS. In one embodiment, the MDS is relapsed or refractory MDS. In one embodiment, the MDS is relapsed or refractory higher risk MDS. In one embodiment, MDS is refractory anemia (RA); RA with ringed sideroblasts (RARS); RA with excess of blasts (RAEB); refractory cytopenia with multilineage dysplasia (RCMD), refractory cytopenia with unilineage dysplasia (RCUD); unclassifiable myelodysplastic syndrome (MDS-U), myelodysplastic syndrome associated with an isolated del(5q) chromosome abnormality, therapy-related myeloid neoplasms or chronic myelomonocytic leukemia (CMML). In some embodiments, the MDS is very low risk, low risk, intermediate risk, high risk or very high risk MDS. In one embodiment, the MDS is very low risk. In another embodiment, the MDS is low risk. In another embodiment, the MDS is intermediate risk. In another embodiment, the MDS is high risk. In another embodiment, the MDS is very high risk MDS. In some embodiments, the MDS is primary or de novo MDS. In other embodiments, the MDS is secondary MDS.

In one embodiment, provided herein are methods of treating, preventing, managing, and/or ameliorating MDS by administering Compound 1 to a subject in a dose of about 0.1 mg to about 20 mg. In one embodiment, provided herein are methods of treating, preventing, managing, and/or ameliorating relapsed or refractory MDS, including high risk relapsed or refractory MDS, by administering Compound 1 to a subject in a dose of about 0.1 mg to about 20 mg. In one embodiment, provided herein are methods of treating MDS by administering Compound 1 to a subject in a dose of about 0.1 mg to about 20 mg. In one embodiment, provided herein are methods of treating relapsed or refractory MDS, including high risk relapsed or refractory MDS, by administering Compound 1 to a subject in a dose of about 0.1 mg to about 20 mg. In one embodiment, provided herein are methods of treating, preventing, managing, and/or ameliorating MDS by administering Compound 1 to a subject in a cycle, wherein the cycle comprises administering Compound 1 in a dose of about 0.1 mg to about 20 mg on days 1 to 5, 1 to 7, 1 to 10, or 1 to 14, of a 28 day cycle. In one embodiment, provided herein are methods of treating, preventing, managing, and/or ameliorating relapsed or refractory MDS, including high risk relapsed or refractory MDS, by administering Compound 1 to a subject in a cycle, wherein the cycle comprises administering Compound 1 in a dose of about 0.1 mg to about 20 mg on days 1 to 5, 1 to 7, 1 to 10, or 1 to 14, of a 28 day cycle. In one embodiment, provided herein are methods of treating, preventing, managing, and/or ameliorating MDS by administering Compound 1 to a subject in a cycle, wherein the cycle comprises administering Compound 1 in a dose of about 0.1 mg to about 20 mg on days 1 to 5, of a 28 day cycle. In one embodiment, provided herein are methods of treating, preventing, managing, and/or ameliorating relapsed or refractory MDS, including high risk relapsed or refractory MDS, by administering Compound 1 to a subject in a cycle, wherein the cycle comprises administering Compound 1 in a dose of about 0.1 mg to about 20 mg on days 1 to 5, of a 28 day cycle. In one embodiment, provided herein are methods of treating, preventing, managing, and/or ameliorating MDS by administering Compound 1 to a subject in a cycle, wherein the cycle comprises administering Compound 1 in a dose of about 0.1 mg to about 20 mg on days 1 to 7, of a 28 day cycle. In one embodiment, provided herein are methods of treating, preventing, managing, and/or ameliorating relapsed or refractory MDS, including high risk relapsed or refractory MDS, by administering Compound 1 to a subject in a cycle, wherein the cycle comprises administering Compound 1 in a dose of about 0.1 mg to about 20 mg on days 1 to 7, of a 28 day cycle. In one embodiment, provided herein are methods of treating, preventing, managing, and/or ameliorating MDS by administering Compound 1 to a subject in a cycle, wherein the cycle comprises administering Compound 1 in a dose of about 0.1 mg to about 20 mg on days 1 to 10, of a 28 day cycle. In one embodiment, provided herein are methods of treating, preventing, managing, and/or ameliorating relapsed or refractory MDS, including high risk relapsed or refractory MDS, by administering Compound 1 to a subject in a cycle, wherein the cycle comprises administering Compound 1 in a dose of about 0.1 mg to about 20 mg on days 1 to 10, of a 28 day cycle. In one embodiment, provided herein are methods of treating, preventing, managing, and/or ameliorating MDS by administering Compound 1 to a subject in a cycle, wherein the cycle comprises administering Compound 1 in a dose of about 0.1 mg to about 20 mg on 1 to 14, of a 28 day cycle. In one embodiment, provided herein are methods of treating, preventing, managing, and/or ameliorating relapsed or refractory MDS, including high risk relapsed or refractory MDS, by administering Compound 1 to a subject in a cycle, wherein the cycle comprises administering Compound 1 in a dose of about 0.1 mg to about 20 mg on 1 to 14, of a 28 day cycle.

In one embodiment, provided herein are methods of treating MDS by administering Compound 1 to a subject in a dose of about 0.1 mg to about 20 mg in a cycle, wherein the cycle comprises administering Compound 1 on days 1, 4, 8, 11, 15, 18, 22, 25 of a 28 day cycle. In one embodiment, provided herein are methods of treating relapsed or refractory MDS, including high risk relapsed or refractory MDS, by administering Compound 1 to a subject in a dose of about 0.1 mg to about 20 mg in a cycle, wherein the cycle comprises administering Compound 1 on days 1, 4, 8, 11, 15, 18, 22, 25 of a 28 day cycle. In one embodiment, provided herein are methods of treating MDS by administering Compound 1 to a subject in a dose of about 0.1 mg to about 20 mg in a cycle, wherein the cycle comprises administering Compound 1 on days 1, 4, 8, 11, 15, 18 of a 28 day cycle. In one embodiment, provided herein are methods of treating relapsed or refractory MDS, including high risk relapsed or refractory MDS, by administering Compound 1 to a subject in a dose of about 0.1 mg to about 20 mg in a cycle, wherein the cycle comprises administering Compound 1 on days 1, 4, 8, 11, 15, 18 of a 28 day cycle. In one embodiment, provided herein are methods of treating MDS by administering Compound 1 to a subject in a dose of about 0.1 mg to about 20 mg in a cycle, wherein the cycle comprises administering Compound 1 on days 1, 4, 8, 11 of a 28 day cycle. In one embodiment, provided herein are methods of treating relapsed or refractory MDS, including high risk relapsed or refractory MDS, by administering Compound 1 to a subject in a dose of about 0.1 mg to about 20 mg in a cycle, wherein the cycle comprises administering Compound 1 on days 1, 4, 8, 11 of a 28 day cycle.

In some such embodiments, provided herein are methods of treating, preventing, managing, and/or ameliorating MDS comprising administering Compound 1 a dose of about 0.1 mg/day, 0.2 mg/day, 0.4 mg/day, 0.5 mg/day, 0.8 mg/day, 1 mg/day, 1.5 mg/day, 2 mg/day, 3 mg/day, 6 mg/day, 9 mg/day and 12 mg/day.

Further provided herein are methods for achieving one or more clinical endpoints associated with AML and/or MDS comprising administering a therapeutically effective amount of Compound 1 to a patient in need thereof.

In certain embodiments, the methods provided herein increase the overall survival (OS), complete remission rate (CRR), objective response rate (ORR), time to progression, relapse free survival (RFS), progression-free survival (PFS) event-free survival, duration of remission, duration of response, and/or time to remission/response in a patient population having AML treated with an effective amount of Compound 1, when compared to a patient population not treated with Compound 1. In certain embodiments, the methods provided herein increase the overall survival (OS), complete remission rate (CRR), objective response rate (ORR), time to progression, relapse free survival (RFS), progression-free survival (PFS) event-free survival, duration of remission, duration of response, time to remission/response, and/or transfusion independence in a patient population having AML treated with an effective amount of Compound 1, when compared to a patient population not treated with Compound 1.

In one embodiment, the methods provided herein increase the overall survival (OS) in a patient population having AML treated with an effective amount of Compound 1, when compared to a patient population not treated with Compound 1.

In one embodiment, the methods provided herein increase the complete remission rate (CRR) in a patient population having AML treated with an effective amount of Compound 1, when compared to a patient population not treated with Compound 1.

In one embodiment, the methods provided herein increase the objective response rate (ORR) in a patient population having AML treated with an effective amount of Compound 1, when compared to a patient population not treated with Compound 1.

In one embodiment, the methods provided herein increase the time to progression in a patient population having AML treated with an effective amount of Compound 1, when compared to a patient population not treated with Compound 1.

In one embodiment, the methods provided herein increase the relapse free survival (RFS) in a patient population having AML treated with an effective amount of Compound 1, when compared to a patient population not treated with Compound 1.

In one embodiment, the methods provided herein increase the progression-free survival (PFS) in a patient population having AML treated with an effective amount of Compound 1, when compared to a patient population not treated with Compound 1.

In one embodiment, the methods provided herein increase the event-free survival in a patient population having AML treated with an effective amount of Compound 1, when compared to a patient population not treated with Compound 1.

In one embodiment, the methods provided herein increase the duration of remission in a patient population having AML treated with an effective amount of Compound 1, when compared to a patient population not treated with Compound 1.

In one embodiment, the methods provided herein increase the duration of response in a patient population having AML treated with an effective amount of Compound 1, when compared to a patient population not treated with Compound 1.

In one embodiment, the methods provided herein increase the time to remission/response in a patient population having AML treated with an effective amount of Compound 1, when compared to a patient population not treated with Compound 1.

In one embodiment, the methods provided herein increase the transfusion independence in a patient population having AML treated with an effective amount of Compound 1, when compared to a patient population not treated with Compound 1.

In certain embodiments, the methods provided herein increase the overall survival (OS), complete remission rate (CRR), objective response rate (ORR), time to progression, relapse free survival (RFS), progression-free survival (PFS) event-free survival, duration of remission, duration of response, and/or time to remission/response in a patient population having MDS treated with an effective amount of Compound 1, when compared to a patient population not treated with Compound 1.

In one embodiment, the methods provided herein increase the overall survival (OS) in a patient population having MDS treated with an effective amount of Compound 1, when compared to a patient population not treated with Compound 1.

In one embodiment, the methods provided herein increase the complete remission rate (CRR) in a patient population having MDS treated with an effective amount of Compound 1, when compared to a patient population not treated with Compound 1.

In one embodiment, the methods provided herein increase the objective response rate (ORR) in a patient population having MDS treated with an effective amount of Compound 1, when compared to a patient population not treated with Compound 1.

In one embodiment, the methods provided herein increase the time to progression in a patient population having MDS treated with an effective amount of Compound 1, when compared to a patient population not treated with Compound 1.

In one embodiment, the methods provided herein increase the relapse free survival (RFS) in a patient population having MDS treated with an effective amount of Compound 1, when compared to a patient population not treated with Compound 1.

In one embodiment, the methods provided herein increase the progression-free survival (PFS) in a patient population having MDS treated with an effective amount of Compound 1, when compared to a patient population not treated with Compound 1.

In one embodiment, the methods provided herein increase the event-free survival in a patient population having MDS treated with an effective amount of Compound 1, when compared to a patient population not treated with Compound 1.

In one embodiment, the methods provided herein increase the duration of remission in a patient population having MDS treated with an effective amount of Compound 1, when compared to a patient population not treated with Compound 1.

In one embodiment, the methods provided herein increase the duration of response in a patient population having MDS treated with an effective amount of Compound 1, when compared to a patient population not treated with Compound 1.

In one embodiment, the methods provided herein increase the time to remission/response in a patient population having MDS treated with an effective amount of Compound 1, when compared to a patient population not treated with Compound 1.

In certain embodiment, the ORR includes all responses of complete remission (CR) (i.e., morphologic leukemia-free state, morphologic CR, cytogenetic CR, molecular CR, and morphologic CR with incomplete blood recovery), and partial remission.

### 6.4 Cycling Therapy/Dosages

In the methods provided herein, a therapeutically effective amount of Compound 1 can be cyclically administered to a patient in need thereof independent of the cancer treated. Cycling therapy can reduce the development of resistance to one or more of the therapies, avoid or reduce the side effects of one of the therapies, and/or improve the efficacy of the treatment.

In one embodiment, a therapeutically effective amount of Compound 1 is administered in a treatment cycle which includes an administration period of up to 5 days followed by a rest period. In one embodiment, the treatment cycle includes an administration period of 7 days followed by a rest period. In one embodiment, the treatment cycle includes an administration period of up to 10 days followed by a rest period. In one embodiment, the treatment cycle includes an administration period of up to 14 days followed by a rest period. In one embodiment, the rest period is about 28 days.

In one embodiment, the treatment cycle includes an administration of a therapeutically effective amount of Compound 1 on days 1 to 5 of a 28 day cycle. In another embodiment, the treatment cycle includes an administration of Compound 1 on days 1 to 7 of a 28 day cycle. In another embodiment, the treatment cycle includes an administration of Compound 1 on days 1 to 10 of a 28 day cycle. In another embodiment, the treatment cycle includes an administration of Compound 1 on days 1 to 14 of a 28 day cycle.

In one embodiment, the treatment cycle includes an administration of a therapeutically effective amount of Compound 1 on days 1, 4, 8, 11, 15, 18, 22 and 25 of a 28 day cycle. In another embodiment, the treatment cycle includes an administration of Compound 1 on days 1, 4, 8, 11, 15 and 18 of a 28 day cycle. In another embodiment, the treatment cycle includes an administration of Compound 1 on days 1, 4, 8 and 11 of a 28 day cycle.

Any treatment cycle described herein can be repeated for at least 2, 3, 4, 5, 6, 7, 8, or more cycles. In certain instances, the cycling therapy is not limited to the number of cycles, and the therapy is continued until disease progression. Cycles, can in certain instances, include varying the duration of administration periods and/or rest periods described herein.

In one embodiment the treatment cycle includes administering Compound 1 at a dosage amount of about 0.05 mg/day to about 20 mg/day, from about 0.1 mg/day to about 15 mg/day, administered once per day.

In one embodiment the treatment cycle includes administering Compound 1 at a dosage amount of about 0.1 mg/day, 0.2 mg/day, 0.4 mg/day, 0.5 mg/day, 0.8 mg/day, 1 mg/day, 1.5 mg/day, 2 mg/day, 3 mg/day, 6 mg/day, 9 mg/day and 12 mg/day administered once per day.

Compound 1 can be administered at the same amount for all administration periods in a treatment cycle. Alternatively, in one embodiment, the compound is administered at different doses in the administration periods.

### 6.5 Combination Therapy

In certain embodiments, the methods provided herein comprise administration of a therapeutically effective amount of Compound 1 in combination with a therapeutically effective amount of other therapeutic agents.

In one embodiment, provided herein is a method of treating, preventing, or managing AML or MDS, comprising administering to a patient a therapeutically effective amount of Compound 1 in a cycling therapy as provided herein in combination with a therapeutically effective amount of one or more second active agents, and optionally in combination with radiation therapy, blood transfusions, biological or immunotherapy, or surgery. Examples of second active agents are disclosed herein.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a patient with a disease or disorder. A first therapy (e.g., a prophylactic or therapeutic agent such as Compound 1 provided herein), can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to the subject. Triple therapy is also contemplated herein.

In certain embodiments, administration of a therapeutically effective amount of Compound 1 and one or more second active agents to a patient can occur simultaneously or sequentially by the same or different routes of administration. The suitability of a particular route of administration employed for a particular active agent will depend on the active agent itself (e.g., whether it can be administered orally without decomposing prior to entering the blood stream) and the cancer being treated.

The route of administration of Compound 1 is independent of the route of administration of a second therapy. Thus, in accordance with these embodiments, Compound 1 is administered intravenously, and the second therapy can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form. In one embodiment, Compound 1 and a second therapy are administered by the same mode of administration, by IV. In another embodiment, Compound 1 is administered by one mode of administration, e.g., by IV, whereas the second agent (an anticancer agent) is administered by another mode of administration, e.g., orally.

In one embodiment, the second active agent is administered intravenously or subcutaneously and once or twice daily in an amount of from about 1 to about 1000 mg, from about 5 to about 500 mg, from about 10 to about 350 mg, or from about 50 to about 200 mg. The specific amount of the second active agent will depend on the specific agent used, the type of disease being treated or managed, the severity and stage of disease, and the amount of Compound 1 and any optional additional active agents concurrently administered to the patient.

In some embodiment, the components of the combination therapies described herein are cyclically administered to a patient. In another embodiment, a second active agent is co-administered in a cyclic administration with the combination therapies provided herein. Cycling therapy involves the administration of an active agent for a period of time, followed by a rest for a period of time, and repeating this sequential administration. Cycling therapy can be performed independently for each active agent (e.g., Compound 1, and/or a second active agent described herein) over a prescribed duration of time. In certain embodiments, the cyclic administration of each active agent is dependent upon one or more of the active agents administered to the subject. In one embodiment, administration of Compound 1 or second active agent described herein fixes the day(s) or duration of administration of each agent. In another embodiment, administration of Compound 1 or second active agent described herein fixes the days(s) or duration of administration of a second active agent.

In some embodiments, Compound 1 and a second active agent described herein are administered continually (e.g., daily, weekly, monthly) without a rest period. Cycling therapy can reduce the development of resistance to one or more of the therapies, avoid, or reduce the side effects of one of the therapies, and/or improve the efficacy of the treatment or therapeutic agent.

In one embodiment, a therapeutically effective amount of Compound 1 is administered as a component of a combination therapy as described herein once daily for days 1 to 5, days 1 to 7, or days 1 to 14, in a 28 days cycle. In one embodiment, a therapeutically effective amount of Compound 1 is administered as a component of a combination therapy as described herein once daily for days 1, 4, 8, 11, 15, 18, 22 and 25 of a 28 days cycle, days 1, 4, 8, 11, 15, and 18 of a 28 days cycle or days 1, 4, 8 and 11 of a 28 days cycle. Such combination therapies comprise administration of a second active agent as described herein prior to, concomitantly with, or subsequent to administration of Compound 1 on one or more days (e.g., on day 1 of cycle 1). In one embodiment, the combination therapy is administered for 1 to 13 cycles of 28 days (e.g., about 12 months). Compound 1 and a second active agent described herein of such a combination can be present at a concentration or amount as set forth herein. In certain embodiments, the second active agent can be administered once daily, once weekly, or once monthly during the cycling therapy. In another embodiment, the second active agent is administered once weekly in combination with a combination therapy described herein.

In one embodiment, a therapeutically effective amount of Compound 1 is administered as a component of a combination therapy as described herein once daily for 7 consecutive days. Such combination therapies comprises administration of a therapeutically effective amount of a second active agent as described herein prior to, concomitantly with, or subsequent to administration of a therapeutically effective amount of Compound 1 on one or more days (e.g., on day 1 of cycle 1). In another embodiment, a therapeutically effective amount of Compound 1 is administered once daily for 7 consecutive days followed by 21 days of rest (e.g., no administration of the compound/discontinuation of treatment) in a 28 days cycle. Such a combination therapy comprises administration of a therapeutically effective amount of a second active agent as described herein prior to, concomitantly with, or subsequent to administration of Compound 1 on one or more days (e.g., on day 1 of cycle 1). In one embodiment, the combination therapy is administered for 1 to 13 cycles of 28 days (e.g., about 3 months). Compound 1 and second active agents as described herein of such a combination can be present at a concentration or amount as set forth herein. In one embodiment the combination therapy comprises administration of a therapeutically effective amount of Compound 1 consecutively for 7 days of a 28 days cycle and administration of a therapeutically effective amount of a second active agent on at least one day of each cycle (e.g., day 1 of cycle 1) in combination with a second active agent administered on at least one day of each cycle. In one embodiment, a therapeutically effective amount of Compound 1 is administered as a component of a combination therapy as described herein once daily on days 1 to 5, 1 to 7, 1 to 10, or 1 to 14, of a 28 day cycle. Such combination therapies comprises administration of a therapeutically effective amount of a second active agent as described herein prior to, concomitantly with, or subsequent to administration of a therapeutically effective amount of Compound 1 on one or more days. In another embodiment, a therapeutically effective amount of Compound 1 is administered once daily on days 1, 4, 8, 11, 15, 18, 22 and 25 of a 28 day cycle. Such a combination therapy comprises administration of a therapeutically effective amount of a second active agent as described herein prior to, concomitantly with, or subsequent to administration of Compound 1 on one or more days. In another embodiment, a therapeutically effective amount of Compound 1 is administered once daily on days 1, 4, 8, 11, 15 and 18 of a 28 day cycle. Such a combination therapy comprises administration of a therapeutically effective amount of a second active agent as described herein prior to, concomitantly with, or subsequent to administration of Compound 1 on one or more days.

In certain embodiments, the second active agent can be administered once daily, once weekly, or once monthly during the cycling therapy. In another embodiment, the second active agent is administered once weekly in combination with a combination therapy described herein.

A compound for use in combination therapies described herein can independently be administered once daily (QD), or divided into multiple daily doses such as twice daily (BID), three times daily (TID), and four times daily (QID) as part of a combination therapy described herein. In addition, the administration can be continuous (i.e., daily for consecutive days or every day), intermittent, e.g., in cycles (i.e., including days, weeks, or months of rest without drug). As used herein, the term “daily” is intended to mean that a therapeutic agent is administered once or more than once each day, for example, for a period of time. The term “continuous” is intended to mean that a therapeutic agent is administered daily for an uninterrupted period of at least 10 days to 52 weeks. The term “intermittent” or “intermittently” as used herein is intended to mean stopping and starting at either regular or irregular intervals. For example, intermittent administration of a compound for use in combination therapies described herein can be administered for one to six days per week, administration in cycles (e.g., daily administration for two to eight consecutive weeks, then a rest period with no administration for up to one week), or administration on alternate days. The term “cycling” as used herein is intended to mean that a therapeutic agent is administered daily or continuously but with a rest period.

In certain embodiments, a compound for use in combination therapies described herein is administered once per day from one day to six months, from one week to three months, from one week to four weeks, from one week to three weeks, or from one week to two weeks. In certain embodiments, a compound for use in combination therapies described herein is administered once per day for one week, two weeks, three weeks, or four weeks. In one embodiment, a compound for use in combination therapies described herein is administered once per day for one week. In another embodiment, a compound for use in combination therapies described herein is administered once per day for two weeks. In yet another embodiment, a compound for use in combination therapies described herein is administered once per day for three weeks. In still another embodiment, a compound for use in combination therapies described herein is administered once per day for four weeks.

One or more second active ingredients or agents can be used together with Compound 1 in the methods and compositions provided herein. Second active agents can be large molecules (e.g., proteins) or small molecules (e.g., synthetic inorganic, organometallic, or organic molecules).

Examples of second active agents include, but are not limited to, cytarabine, daunorubicin, idarubicin, midostaurin, cladribine, gemtuzumab ozogamicin, fludarabine, mitoxantrone, gilteritinib, glasdegib, and venetoclax.

It will be appreciated that every suitable combination of the compounds provided herein with one or more of the aforementioned compounds and optionally one or more further pharmacologically active substances is contemplated herein.

### 6.6 Patient Population

In certain embodiments of the methods provided herein, the subject is an animal, for example, a mammal, or a non-human primate. In a particular embodiment, the subject is a human. The subject can be a male or female subject.

In one embodiment, subjects for the methods provided herein include human patients, for example, those who have been diagnosed with acute myeloid leukemia, including relapsed or refractory acute myeloid leukemia. In one embodiment, subjects for the methods provided herein include human MDS patients, including patients with relapsed or refractory MDS.

In one embodiment, the subject has relapsed or refractory AML and R/R HR-MDS as defined by the WHO criteria who have failed or are ineligible for all available therapies which may provide clinical benefit. In one embodiment, the subject has no known TP53 mutation or loss of heterozygosity for TP53 or chromosome p17 as determined by NGS, polymerase chain reaction, fluorescence in situ hybridization (FISH), or karyotype analysis obtained during standard of care pre-study evaluations. In one embodiment, the subject has relapsed or refractory AML and R/R HR-MDS as defined by the WHO criteria who have failed or are ineligible for all available therapies which may provide clinical benefit, and have no known TP53 mutation or loss of heterozygosity for TP53 or chromosome p17 as determined by NGS, polymerase chain reaction, fluorescence in situ hybridization (FISH), or karyotype analysis obtained during standard of care pre-study evaluations.

In some embodiments, the subject is 18 years or older. In some embodiments, the subject is more than 18, 25, 35, 40, 45, 50, 55, 60, 65, or 70 years old. In other embodiments, the subject is less than 65 years old.

In some embodiments, the subject is treated based on the Eastern Cooperative Oncology Group (ECOG) performance status score of the subject for leukemia. In some embodiments, the subject has an ECOG performance status score of 0 to 2. In some embodiments, the subject has an ECOG performance status score of 0. In some embodiments, the subject has an ECOG performance status score of 1. In other embodiments, the subject has an ECOG performance status score of 2.

In some embodiments, the subject is treated based on the Eastern Cooperative Oncology Group (ECOG) performance status score of the subject for myelodysplastic syndrome (MDS). In some embodiments, the subject has an ECOG performance status score of 0 to 2. In some embodiments, the subject has an ECOG performance status score of 0. In some embodiments, the subject has an ECOG performance status score of 1. In other embodiments, the subject has an ECOG performance status score of 2.

In certain embodiments, the methods provided herein encompass the treatment of a subject in which at least 4 weeks (from first dose of Compound 1) have elapsed from donor lymphocyte infusion (DLI) without conditioning.

In certain embodiments, the methods provided herein encompass the treatment of a subject who has the following screening laboratory values:

Corrected serum Ca or free (ionized) serum Ca within normal limits (WNL).

Corrected Ca (mg/dL)=Total Ca (mg/dL)−0.8 (albumin [g/dL]−4)

Total White Blood Cell count (WBC) <25×109/L prior to first infusion. Prior leukapheresis and/or prior or concurrent treatment with hydroxyurea to achieve this level are allowed.

Potassium and magnesium within normal limits or correctable with supplements.

Aspartate aminotransferase/serum glutamic oxaloacetic transaminase (AST/SGOT) or alanine aminotransferase/serum glutamate pyruvic transaminase (ALT/SGPT)≤2.5×Upper Limit of Normal (ULN).

Uric acid≤7.5 mg/dL (446 μmol/L). Prior and/or concurrent treatment with hypouricemic agents (eg, allopurinol, rasburicase) are allowed.

Serum bilirubin≤1.5×ULN.

Estimated serum creatinine clearance of ≥60 mL/min using the Cockcroft-Gault equation.

INR<1.5×ULN and PTT<1.5×ULN.

In other embodiments, the methods encompass treating subjects who have been previously treated or are currently being treated for AML. For example, the subject may have been previously treated or are currently being treated with a standard treatment regimen for AML. The subject may have been treated with any AML treatment regimen prescribed by the practitioner of skill in the art. In certain embodiments, the subject has been previously treated with at least one induction/reinduction or consolidation AML regimen. In some embodiments, the subject has undergone autologous bone marrow transplantation or stem cell transplantation as part of a consolidation regimen.

In certain embodiments, the subject has no clinical symptoms suggesting active central nervous system (CNS) leukemia or known CNS leukemia.

In certain embodiments, the subject does not have immediately life-threatening, severe complications of AML such as disseminated/uncontrolled infection, uncontrolled bleeding, and/or uncontrolled disseminated intravascular coagulation.

In certain embodiments, the subject does not have impaired cardiac function or clinically significant cardiac diseases.

In some embodiments, the subject has not undergone prior autologous hematopoietic stem cell transplant 3 months or less than 3 months prior to treatment of Compound 1 according to the methods provided herein.

In some embodiments, the subject has not undergone prior allogeneic hematopoietic stem cell transplant (HSCT) with either standard or reduced intensity conditioning less than 6 months prior to starting treatment with Compound 1 according to the methods provided herein.

In some embodiments, the subject is not on systemic immunosuppressive therapy post HSCT, or with clinically significant graft-versus-host disease (GVHD).

In some embodiments, the subject has not undergone prior systemic cancer-directed treatments or investigational modalities less than five half lives or 4 weeks prior to starting treatment of Compound 1, whichever is shorter. In some embodiments, the subject has received hydroxyurea treatment.

In some embodiments, the subject has not undergone a major surgery less than two weeks prior to starting treatment of Compound 1.

In some embodiments, the subject has no known HIV infection. In some embodiments, the subject has no known chronic, active hepatitis B or C (HBV/HCV) infection.

In some embodiments, the subject is not undergoing treatment with chronic, therapeutic dosing of anti-coagulants (eg, warfarin, low molecular weight heparin, Factor Xa inhibitors). In some embodiments, the subject has no history of concurrent second cancers requiring active, ongoing systemic treatment.

In certain embodiments, the subject has no disorders or conditions disrupting normal calcium homeostasis or preventing calcium supplementation.

Because subjects with cancer have heterogeneous clinical manifestations and varying clinical outcomes, the treatment given to a patient may vary, depending on his/her prognosis. The skilled clinician will be able to readily determine without undue experimentation specific secondary agents, types of surgery, and types of non-drug based standard therapy that can be effectively used to treat an individual subject with cancer.

### 6.7 Evaluation of Activity

Standard physiological, pharmacological and biochemical procedures are available for testing the compounds to identify those that possess the desired anti-proliferative activity.

Such assays include, for example, biochemical assays such as binding assays, radioactivity incorporation assays, as well as a variety of cell based assays.

Embodiments provided herein may be more fully understood by reference to the following examples. These examples are meant to be illustrative of pharmaceutical compositions and dosage forms provided herein, but are not in any way limiting.

### 7. Examples

The following Examples are presented by way of illustration, not limitation. The following abbreviations are used in descriptions and examples.

| Abbreviation | Explanation |
|---|---|
| AE | Adverse event |
| ALT | Alanine aminotransferase (SGPT) |
| AML | Acute Myeloid Leukemia |
| AST | Aspartate aminotransferase (SGOT) |
| AUC | Area under the curve |
| AUC₀₋₂₄ | Area under the plasma concentration time- |
| | curve from time 0 to 24 hours postdose |
| AUC_{LST} | AUC from the time of dosing to the last |
| | measurable positive concentration |
| BAX | BCL-2 associated X protein |
| BIW | Twice a week |
| BLRM | Bayesian logistic regression model |
| BM | Bone marrow |
| CBC | Complete blood count |
| CEBPα | CCAAT enhancer-binding protein α |
| cCRR | Combined complete response rate |
| CK1α | Casein kinase 1α |
| CL | Clearance |
| CL/F | Apparent total clearance of the drug from |
| | plasma after oral administration |
| CD | Cluster of differentiation |
| Cₘₐₓ | Maximum plasma drug concentration |
| CNS | Central nervous system |
| CR | Complete remission |
| CRi | CR with incomplete hematologic recovery |
| CRiR | CRi rate |
| CRBN | Cereblon |
| CRh | CR with partial hematologic recovery |
| CRR | Complete remission rate |
| CRR MRD- | Minimal residual disease negative CRR |
| del(5q) | deletion of chromosome 5q |
| DL | Dose level |
| DLT | Dose-limiting toxicity |
| DNA | Deoxyribonucleic acid |
| DOR | Duration of response |
| ECG | Electrocardiogram |
| ECHO | Echocardiogram |
| ECOG PS | Eastern Cooperative Oncology |
| | Group Performance Status |
| EEA | European Economic Area |
| EFS | Event-free survival |
| EOT | End of treatment |
| EWOC | Escalation with overdose control |
| FLT3 | fms-like tyrosine kinase 3 |
| G-CSF | Granulocyte-colony stimulating factor |
| GLP | Good Laboratory Practice |
| GM-CSF | Granulocyte-macrophage stimulating factor. |
| GVHD | graft-versus-host disease |
| HCV | Hepatitis C virus C |
| HI | Hematologic improvement |
| HIR | Hematologic improvement rate |
| HIV | Human immunodeficiency virus |
| HNSTD | Highest non-severely toxic dose |
| HR-MDS | Higher risk myelodysplastic syndrome |
| HSCT | Hematopoietic stem cell transplant |
| IC50 | Fifty percent inhibitory concentrations |
| IPSS-R | Revised international prognostic scoring system |
| IV | Intravenous |
| IWG | International Working Group |
| LVEF | Left ventricular ejection fraction |
| MDM2 | Murine double minute 2 homolog |
| MDS | Myelodysplastic syndrome |
| MIC-1 | Macrophage inhibitory cytokine 1 |
| MLFS | Morphologic Leukemia-free State |
| MLFSR | MLFS rate |
| mOS | Median overall survival |
| MRD | Minimal residual disease |
| MTD | Maximum tolerated dose |
| NOAEL | no observed adverse effect level |
| NPM1 | Nucleophosmin 1 |
| ORR | Overall response rate |
| OS | Overall survival |
| p21 | cyclin-dependent kinase inhibitor 1 |
| p53 | tumor protein p53 |
| PB | Peripheral blood |
| PD | Pharmacodynamic |
| PFS | Progression-free survival |
| pH3s10 | phospho-histone H3 S10 |
| PK | Pharmacokinetics |
| PPP | Pregnancy prevention plan |
| PR | Partial remission |
| PRR | PR rate |
| PT | Prothrombin time |
| PTT | Partial thromboplastin time |
| PUMA | p53 upregulated modulator of apoptosis |
| QD | Once daily |
| RBC | Red blood cell |
| RECIST | Response Evaluation Criteria in Solid Tumors |
| RFS | Relapse-free survival |
| RNA | Ribonucleic acid |
| RP2D | Recommended Phase 2 dose |
| R/R | Relapsed or refractory |
| RT-PCR | Real-time reverse transcription |
| | polymerase chain reaction |
| SAE | Serious adverse event |
| SARS-CoV-2 | Severe acute respiratory syndrome coronavirus 2 |
| SD | Stable disease |
| SDR | SD rate |
| SGOT | Serum glutamic oxaloacetic transaminase |
| SGPT | Serum glutamic pyruvic transaminase |
| STD | Severely toxic dose |
| SUSAR | Suspected unexpected serious adverse reaction |
| t1/2 | Half-life |
| TEAEs | treatment-emergent adverse events |
| TLS | Tumor lysis syndrome |
| Tmax | Time to peak (maximum) plasma concentration |
| TSH | Thyroid-stimulating hormone |
| TP53 | Tumor protein 53 |
| ULN | Upper limit of normal |
| WBC | White blood cell count |
| WT | Wild-type |

### Example 1: In Vitro Pharmacology

Compound 1 displayed potent and broad cell autonomous activity against TP53 WT AML cell lines with distinct French-American-British subtypes and carrying various onco-driver mutations, while AML cell lines with TP53 loss or mutation were insensitive to Compound 1 treatment. Of the 13 TP53 WT AML cell lines tested, 8 were sensitive to Compound 1, with fifty percent inhibitory concentrations (IC₅₀) values ranging from 15 to 500 nM. The remaining 5 cell lines were either medium-sensitive or completely resistant to Compound 1; the unresponsiveness to Compound 1 treatment was associated with inadequate accumulation of p53 protein and/or its transcriptional targets BAX and PUMA.

The cytotoxic effect of Compound 1 was also demonstrated on leukemic cells from TP53 WT AML patients using the PharmaFlow test at Vivia, an assay predictive of clinical response to standard-of-care chemotherapy in AML (Martinez-Cuadron et al. A precision medicine test predicts clinical response after idarubicin and cytarabine induction therapy in AML patients, Leuk Res 2019;76, 1-10).

In 5 out of 9 AML patient BM samples, leukemic cells were sensitive to Compound 1 treatment with IC₅₀ values ranging from 22 nM to 412 nM. Normal T lymphocytes in BM samples from all but 1 patient were noticeably more resistant to Compound 1. In contrast, RG7388 (idasanutlin), a murine double minute 2 homolog (MDM2) inhibitor which has been tested in a Phase 3 clinical trial for the treatment of R/R AML, exhibited pan-cytotoxicity in leukemic cells and T-lymphocytes from the same type of AML patients, indicating that Compound 1 might have a better therapeutic index than RG7388.

### Example 2: Ex Vivo Pharmacology

Ex vivo activity of Compound 1 was evaluated in normal and leukemic progenitors from BM samples of 25 AML patients using colony forming assays. Formation of colony forming unit-granulocyte macrophage (CFU-GM) colonies was inhibited by Compound 1 in most of the TP53 WT AML samples (14 of 18; 77.8%) compared to vehicle control. These results suggest selective efficacy of Compound 1 in reducing the number of leukemic progenitor cells over normal hematopoietic progenitors. Additionally, when these BM cells were cultured for 5 days in liquid culture, the total blast numbers were deeply decreased due to cell cycle arrest and increased apoptosis in response to Compound 1 in all TP53 WT AML samples tested compared to vehicle control. All samples showed IC₅₀ in the nanomolar range. The effect of Compound 1 on CK1α degradation, p53 accumulation, and activation is concentration- and time-dependent. In MV4-11 AML cell line, treatment with 0.3 μM Compound 1 for 12 hours followed by Compound 1 washout led to rapid CK1α protein recovery and p53 protein destabilization; however, the p53 downstream events including induction of p21, phosphor-Ser139-Histone H2A.X, and caspase 3 cleavage persisted for at least 10 hours, suggesting that intermittent dosing with Compound 1 might be sufficient to achieve maximal anti-AML activity.

CD34⁺ cells isolated from BM samples of 3 TP53 WT AML donors and 3 healthy donors were treated with increasing concentrations of Compound 1 in liquid culture at times ranging from 4 hours to 5 days. A differential effect on apoptosis induction was observed in primary AML blasts and leukemic stem cells compared with normal hematopoietic progenitor and stem cells. Cycle arrest was observed primarily in normal cells, while an apoptotic outcome was detected in leukemic cells. Acute myeloid leukemia blasts sustained CK1α degradation up to 5 days in culture as assessed by fluorescence-activated cell sorting (FACS) analysis. Strikingly, p53 stabilization was sustained longer in leukemic cells (up to 48 hours) compared with normal BM cells (for 24 hours). These results suggest that 48 hours of p53 stabilization may be required in AML primary samples to induce apoptosis in leukemic cells.

In summary, Compound 1 inhibited the colony forming ability of leukemic progenitors in most AML patient-derived TP53 WT BM samples analyzed; induced apoptosis and cell cycle arrest in liquid culture with IC₅₀ values in the nanomolar range, and these effects were mediated by CK1α degradation and p53 stabilization. By comparison, Compound 1 had less effect on normal hematopoietic progenitors from healthy donors, suggesting that Compound 1 may be an effective and selective therapy for the treatment of AML.

### Example 3: In Vivo Pharmacology

The CK1α degradation activity and subsequent antitumor activity of Compound 1 in vivo was tested in two TP53 WT AML cell line-derived xenograft models. The depth and duration of CK1α degradation was dose-dependent. Compound 1 significantly reduced tumor burden and prolonged the survival of mice with disseminated MV-4-11 AML in a dose-, schedule- and duration-dependent manner. The animals dosed for longer duration showed better reduction in tumor burden and prolonged survival.

Based on single dose pharmacokinetic (PK)/pharmacodynamics (PD) and efficacy studies with the MV-4-11 AML xenograft model, nearly 90% degradation of CK1α at 2 hours post-dose and 50% suppression through 16 hours a day was maintained when the tumor Compound 1 concentration was >0.1 μM. Maintenance of >0.1 μM Compound 1 in tumor, and this degree of CK1α degradation with daily dosing at 3 mg/kg, led to a 73-99% reduction in tumor burden with significant survival benefit.

Additional studies with various doses and intermittent (3 days on, 2 days off; 3 days on, 4 days off; 5 days on, 2 days off) dosing schedules in the MV-4-11-luc disseminated AML xenograft model also showed schedule-dependent reduction of tumor burden, and prolonged survival. These studies suggest that a dosing schedule of 3-days on/4-days off was as good as 3-days on/2-days off and provided superior leukemia control with daily administration over intermittent dosing. In a subsequent study, MV-4-11-luc disseminated AML xenografts were treated with 2 cycles of fixed doses and varying dosing duration (5/28 days, 7/28 days and 14/28 days). The animals dosed for longer duration showed better reduction in tumor burden with prolonged survival. In Kaplan-Meier survival analysis following cessation dosing, the median survival of the vehicle treated animals was 45.5 days. The animals treated with Compound 1 with 2 cycles of 5/28-days at 50 mg/kg survived significantly (log-rank test) longer than vehicle control with a median survival of 68.5 days (p<0.0001). The median survival of animals treated with Compound 1 with 2 cycles of 7/28-days at 10, 30, and 50 mg/kg was 67.5 (p<0.0001), 70 (p<0.0001), and 70 (p<0.0001) days, respectively. For the animals treated with 2 cycles of 14/28-days at 3, 10 and 30 mg/kg was 69.5 (p<0.0001), 94.5 (p<0.0001) and 152 (p<0.0001) days, respectively. For 30 mg/kg with 14/28-day cycle, 4/10 animals remained tumor free at the termination of the study on day 154.

Overall, treatment with Compound 1 significantly degraded CK1α and activated downstream pathway markers such as p53 and p21 and induced apoptosis as demonstrated by induction of cleaved caspase-3 in AML xenograft tumors. Significant degradation of CK1α and activation of downstream pathways in Compound 1-treated tumors suggests that the observed antitumor activity of Compound 16 was mediated through the degradation of CK1α and activation of p53 and p21.

### Example 4: Safety Pharmacology

Cardiovascular assessments (electrocardiograms [ECGs] and heart rates) were conducted in conscious cynomolgus monkeys as part of the Good Laboratory Practice (GLP)-compliant repeat dose toxicity study. In this study, Compound 1 was administered by oral gavage to Cynomolgus monkeys (5/sex/group) in Groups 1-5 at doses of 0 (0.5% (w/v) Methylcellulose, 0.25% (v/v) Tween 80 in 50 mM citrate buffer pH 3), 0.03, 0.1, 0.1 and 0.3 mg/kg, respectively, intermittently using 2 different dosing regimens. Groups 2 and 3 animals were dosed for 7 consecutive days in each 4-week cycle for 2 cycles (Regimen 1: on Days 1-7 and 29-35), whereas Groups 4 and 5 animals were dosed twice weekly for 4 weeks (Regimen 2: on Days 1, 4, 8, 11, 15, 18, 22 and 25). A combination of both dosing regimens (Days 1-7, 8, 11, 15, 18, 22, 25, and 29-35) was used for administration of the vehicle to the control animals. A 9-lead ECG recording was made on all animal during pretest and on all animals in Groups 4 and 5 on Day 22 (1 hour postdose±15 minutes) and on all animals in Groups 1, 2 and 3 during Week 5 (1 hour postdose±15 minutes) using standard limb leads I, II and III; augmented leads aVR, aVL and aVF as well as chest leads MV₁, MV₂ and MV₃. All ECGs were performed while the unanesthetized animal was sitting in an up-right position in a restraint chair. Qualitative (visual inspection) and quantitative assessments including heart rates and PR, QRS, RR, QT, and QTc intervals were performed. QTc was calculated using Bazett's formula. There were no Compound 1 related effects on heart rate, PR, QRS, QT, and QTc intervals. The sex-combined mean maximum observed plasma concentration values were 31.9 ng/mL at 0.1 mg/kg/day with Regimen 1 on Day 34 and 62.6 ng/mL at 0.3 mg/kg/day with Regimen 2 on Day 15. These values are approximately 80- and 157-fold, respectively, higher than the anticipated maximum plasma drug concentration (Cₘₐₓ)of 0.4 ng/mL at the proposed human Compound 1 starting dose of 0.1 mg/day.

The effect of Compound 1 on the cloned hERG potassium channel expressed in the HEK-293 cell line was also investigated. Compound 1 did not precipitate at concentrations up to 30 μM. In the IonWorks™ Barracuda patch clamp electrophysiology assay, Compound 1 had an IC₅₀ value of >30 μM. The percent inhibition at 30 μM was 19%.

### Example 5: Nonclinical Pharmacokinetics

The systemic clearance of Compound 1 was low and the volume of distribution was high in rats and monkeys. The terminal half-life of Compound 1 was moderate in rats (1.5 to 1.7 hours) and monkeys (2.3 to 4.4 hours). Notable sex differences in PK were not observed in either species. Compound 1 had good oral bioavailability (≥50%) in rats and monkeys. No accumulation was observed in following multiple doses in rats or monkeys.

Plasma protein binding for Compound 1 was high or moderately high in human and nonclinical species, ranging from 89.7% to 99.3%. There was no marked difference in free fraction among human, rat and monkey. Over the concentration ranges tested (25- to 50-fold range) in each species, there was <2-fold change in free fraction. There was no or limited partitioning of Compound 1 into blood cells.

Compound 1 underwent non-enzymatic hydrolysis, oxidation, N-dealkylation, glucuronidation and a combination of these pathways in hepatocytes of human and animal species. Although there were quantitative differences in the formation of Compound 1 metabolites across species, qualitatively all metabolites formed in human hepatocytes were also observed in hepatocytes and plasma of rats and/or monkeys, the two species used for nonclinical safety testing. Following multiple oral administrations of Compound 1 to rats and monkeys, Compound 1 was the predominant component in plasma of both species. The metabolites observed in plasma were similar to those observed in hepatocytes. Enzymatic metabolism of Compound 1 is primarily mediated by CYP3A4/5.

In human liver microsomes, Compound 1 is a weak inhibitor of CYP2C8, CYP2C9, CYP3A4/5 with IC₅₀ values >30 μM, while little (≤25%) to no inhibitory effect was observed on CYP1A2, CYP2A6, CYP2B6, CYP2C19, CYP2D6, and CYP2E1. Compound 1 is not a time-dependent inhibition of CYP1A2, CYP2A6, CYP2B6, CYP2C8, CYP2C9, CYP2C19, CYP2D6, and CYP2E1 following a 30-minute preincubation with NADPH and is a weak time-dependent inhibitor of CYP3A4/5 (<20% at 30 μM). In human hepatocyte culture, Compound 1 treatment up to 50 μM for three consecutive days resulted in no dose-dependent increases in mRNA expression of CYP1A2, CYP2B6 and CYP3A4. These data suggest that clinically relevant drug-drug interactions through CYP inhibition or induction is unlikely when Compound 1 is coadministered with CYP substrates.

Based on allometric scaling of clearance and volume of distribution in non-clinical species, human clearance is predicted to be low at 111 mL/hr/kg (approximately 9% of hepatic blood flow) and volume of distribution is predicted to high at 1.42 L/kg (approximately 2.5-fold of total body water volume). Using the derived PK parameters from allometric scaling and assuming 50% oral bioavailability in humans, the predicted human Cₘₐₓ and area under the plasma concentration time-curve from time 0 to 24 hours postdose (AUC₀₋₂₄) values are 0.4 ng/mL and 5.3 ng·h/mL at the anticipated human starting once daily (QD) dose of 0.1 mg. This projected single dose exposure is approximately 10-fold lower than that at the HNSTD (the highest nonseverely toxic dose, 0.03 mg/kg/day) in cynomolgus monkeys.

### Example 6: Nonclinical Toxicology

### Bacterial Reverse Mutation Test in Salmonella typhimurium and Escherichia coli

Compound 1 was tested in Salmonella typhimurium strains (TA1535, TA1537, TA98, TA100) and Escherichia coli strain WP2 uvrA at a range of concentrations up to 5000 μg/plate (the standard limit concentration for this assay), in the presence and absence of a supplemented rat liver fraction (S9 mix), using the plate incorporation version of the bacterial reverse mutation test. Dimethyl sulfoxide (DMSO) was used as the solvent.

The mean revertant colony counts for the negative controls were within the laboratory historical control range. Bacteria were also incubated with standard positive controls, and the response of the various bacterial strains to these agents confirmed the sensitivity of the test system and the activity of the S9 mix. Therefore, the results from both the negative and positive controls confirmed the validity of the assay.

Incomplete, or absent, background lawns of non-revertant bacteria, or substantial reductions in revertant colony counts, were not obtained following exposure to Compound 1 for any strains, indicating that Compound 1 was non-toxic to the bacteria at the highest concentrations tested. Precipitation was observed in all tester strains in the absence of S9 at a concentration of 5000 μg/plate of Compound 1. No substantial increases in revertant colony numbers were obtained with any of the tester strains, following exposure to Compound 1 at any concentration, in either the presence or absence of S9 mix. Therefore, Compound 1 was considered to be negative for the induction of mutagenicity in this in vitro assay.

Compound 1 did not show any evidence of genotoxic activity in this in vitro mutagenicity assay.

### Micronucleus Assay Using the Human Peripheral Blood Lymphocytes

Compound 1 was tested in the in vitro mammalian cell micronucleus test in human peripheral blood (PB) lymphocytes in the presence and absence of a metabolic activation system, Aroclor-induced rat liver fraction (S9), for 4 hours, and in the absence of S9 for 24 hours. The highest concentration tested was 481 μg/mL. Dimethyl sulfoxide (DMSO) was used as the solvent. For both 4 hour treatment regimes, the highest concentration of test item selected for micronucleus scoring was the highest concentration tested since the observed cytotoxicity (approximately 55% cytotoxicity, based on Cytokinesis-Block Proliferation Index (CBPI) results) was not limiting. For the 24 hour treatment regime, the highest concentration of test item selected for micronucleus scoring was that which produced approximately 55% cytotoxicity, based on CBPI results (120 μu g/mL). In addition, 2 lower concentrations with appropriate concentration and cytotoxicity spacing were chosen for micronucleus scoring in each regime. The negative control results were within the laboratory negative historical control data. The positive controls for clastogenicity (mitomycin C (MMC), cyclophosphamide monohydrate (CP)) and aneugenicity (nocodazole (NOC)) produced statistically significant increases in the incidence of micronucleated binucleate cells compared with the concurrent negative controls. The values from positive controls were also above the 95% upper limit of the historical negative control data, confirming the sensitivity of the test system and the effectiveness of the S9 mix. The assay was therefore considered valid.

Compound 1 did not cause any statistically significant increases in the proportion of micronucleated binucleate cells (MBC) at any experimental point up to the maximum recommended concentration of 1 mM (4 hour regimes) or the limit of cytotoxicity (approximately 55%, 24 hour regime) and none of the treatment groups scored for micronuclei produced an incidence of MBC in excess of the upper 95% observed limit for the laboratory negative historical control range.

Compound 1 did not show any evidence of genotoxic activity in this in vitro test for induction of micronuclei in human peripheral blood lymphocytes in this assay.

### Dose Toxicity Study in Rats

The toxicity and toxicokinetics profile of Compound 1 when administered via oral gavage to rats intermittently for 1 or 2 treatment cycles was studied. Two different dosing regimens of Compound 1 were compared to determine differences in toxicity profile. Additionally, control animals were dosed with a vehicle using a combination of both dosing regimens. Recovery of test article-related effects was evaluated during a 4-week recovery period.

Animals in Regimen 1 were dosed for 7 consecutive days in each 4-week cycle for 2 cycles (0.3 or 3 mg/kg on Days 1-7 and 29-35), whereas animals in Regimen 2 were dosed twice weekly for 4 weeks (0.5 or 5 mg/kg on Days 1, 4, 8, 11, 15, 18, 22 and 25). End of dosing necropsy was performed on the day after the last dose administration. A 4-week recovery period was included to assess reversibility of findings. Administration of Compound 1 was associated with adverse microscopic changes in the BM (decreased cellularity) with correlating hematology changes (decreased red cell mass, leukocytes, and platelets) at 3 mg/kg (Regimen 1) and 5 mg/kg (Regimen 2), and microscopic findings in the male reproductive tract (tubular degeneration and spermatocyte depletion in the testes, cellular debris and reduced sperm in the epididymides) and atrophy in the prostate and mammary (males) glands in all Compound 1-treated groups. Therefore, a no observed adverse effect level (NOAEL) was not identified for the males (both regimens). For Regimen 1, the NOAEL (females) and the severely toxic dose (STD) in 10% of the animals (STD 10) was considered to be 0.3 mg/kg, corresponding to a Cₘₐₓ of 225 ng/mL and an AUC from the time of dosing to the last measurable positive concentration (AUCLST) of 761 ng·hr/mL (sexes combined). For Regimen 2, the NOAEL (females) and the STD 10 was considered to be 0.5 mg/kg, corresponding to a Cₘₐₓ of 263 ng/mL and an AUCLST of 1200 ng·hr/mL (sexes combined).

### Dose Toxicity Study in Monkeys

In a repeat-dose toxicity study in monkeys, Compound 1 was administered by oral gavage intermittently using 2 different dosing regimens. Animals in Regimen 1 were dosed for 7 consecutive days in each 4-week cycle for 2 cycles (0.03 or 0.1 mg/kg on Days 1-7 and 29-35), whereas animals in Regimen 2 were dosed twice weekly for 4 weeks (0.1 or 0.3 mg/kg on Days 1, 4, 8, 11, 15, 18, 22 and 25).

End of dosing necropsy was performed on the day after the last dose administration. A 4-week recovery period was included to assess reversibility of findings. Administration of Compound 1 was associated with mortality at 0.1 mg/kg using Regimen 1 during the recovery phase. Three animals (2 males and 1 female) were euthanized for welfare reasons and one female was found dead due to reduction (marked to severe) in BM cellularity resulting in severe leukopenia and likely bacteremia, as well as in severe thrombocytopenia with disseminated petechial hemorrhage. Similar adverse hematology changes and decreased BM cellularity were observed in animals treated at 0.1 mg/kg and 0.3 mg/kg using Regimen 2. No adverse findings were observed in animals treated at 0.03 mg/kg using Regimen 1. Therefore, the NOAEL and highest non-STD (HNSTD) was considered to be 0.03 mg/kg for Regimen 1, corresponding to a Cₘₐₓ of 9.95 ng/mL and an AUC_{LST} of 51.4 ng·hr/mL (sexes combined). For Regimen 2, a NOAEL was not identified and the HNSTD was considered to be 0.1 mg/kg, corresponding to a Cₘₐₓ of 21.5 ng/mL and an AUC_{LST} of 173 ng·hr/mL (sexes combined).

### Example 7: A Phase 1, Open-Label, Dose-Finding Study of Compound 1 in Subjects with Relapsed or Refractory Acute Myeloid Leukemia or Relapsed or Refractory Higher-Risk Myelodysplastic Syndromes

Indication: Treatment of relapsed or refractory acute myeloid leukemia (R/R AML);
Treatment of relapsed or refractory higher-risk myelodysplastic syndromes (R/R HR-MDS).

### Objectives

### Primary Objectives

To determine the safety and tolerability of Compound 1.

To define the maximum tolerated dose (MTD) and/or the recommended Phase 2 dose (RP2D) and schedule(s) of Compound 1.

### Secondary Objectives

To assess the preliminary efficacy of Compound 1 in R/R AML and R/R HR-MDS.

To characterize the pharmacokinetic (PK) of Compound 1 and Compound 2 in plasma.

### Study Design

This is an open-label, Phase 1, dose escalation and expansion, first in human clinical study of Compound 1 in subjects with R/R AML or in subjects with R/R HR MDS. Both subject populations must not have known TP53 mutations or loss of heterozygosity which is determined locally as a standard of care pre-study evaluations. The Dose Escalation part (Part A) of the study will enroll subjects with R/R AML and R/R HR-MDS and will evaluate the safety and tolerability of escalating doses of Compound 1 administered orally and determine the maximum tolerated dose (MTD) and/or preliminary recommended Phase 2 dose (RP2D) and schedule(s). Throughout the study, final decisions on dose escalation/de-escalation will be made by the safety review committee (SRC).

The expansion part (Part B) will confirm tolerability of the selected doses and schedules and evaluate whether efficacy is in a range that warrants further development. Part B will enroll subjects in two separate cohorts for R/R AML and R/R HR-MDS.

Approximately 40 subjects may be enrolled on Part A (Dose Escalation) in a once daily (QD) and twice a week (BIW) administration schedules. During Part B (Dose Expansion), approximately 20 to 40 response evaluable subjects per cohort may be enrolled with 2 cohorts planned for a total of approximately 80 subjects.

### Part A-Dose Escalation

To limit the number of subjects exposed to sub-therapeutic doses, an accelerated titration approach will be used initially to escalate from the starting dose of 0.1 mg with enrollment of ≥1 subject per dose level (DL). The following observations during dose escalation as per acceleration titration approach will mandate increasing the number of subjects from a minimum of 1 to a minimum of 3 per cohort:
Grade 2 or higher non-hematologic AEs in a single subject unless the event can be clearly be determined to be due to other specified causes.
Grade 4 thrombocytopenia that constitutes a platelet decrease of >50% from Cycle 1 Day 1 in a subject who is not transfusion dependent at baseline.
Grade 4 neutropenia with a decrease of >50% from baseline not explained by disease progression or concomitant use of hydroxyurea (this criteria will exclude subjects who have grade 4 neutropenia at baseline).
Strong pharmacologic effect as evidenced by clearance of peripheral blood blasts or neutrophils (in a subject with no blasts in the blood) during the dosing period.

Following cohort expansion from a minimum of 1 to at least 3 subjects per cohort, observation of a DLT will end the accelerated titration phase and trigger the use of a Bayesian logistic regression model (BLRM) with escalation with overdose control (EWOC) (Babb et al. Cancer phase I clinical trials: efficient dose escalation with overdose control Stat Med. 1998;17(10):1103-20; Neuenschwander et al. Critical aspects of the Bayesian approach to phase I cancer trials Stat Med. 2008;27(13):2420-39) for all subsequent dose escalation steps (a DLT during the single patient cohorts would lead to the same decision to go to multi-patient cohorts and initiate use of BLRM) which will incorporate available prior safety information, update the model parameters after each new cohort of subjects completes Cycle 1, and provide a guidance for Compound 1 dose escalation and estimation of the MTD/RP2D. All patients enrolled in the active dose cohort will be observed throughout the DLT period and the posterior probability of DLT rate exceeding 25% will be estimated before BLRM-based dose selection guidance for subsequent cohorts is provided to the SRC for selection of the next dosing increment.

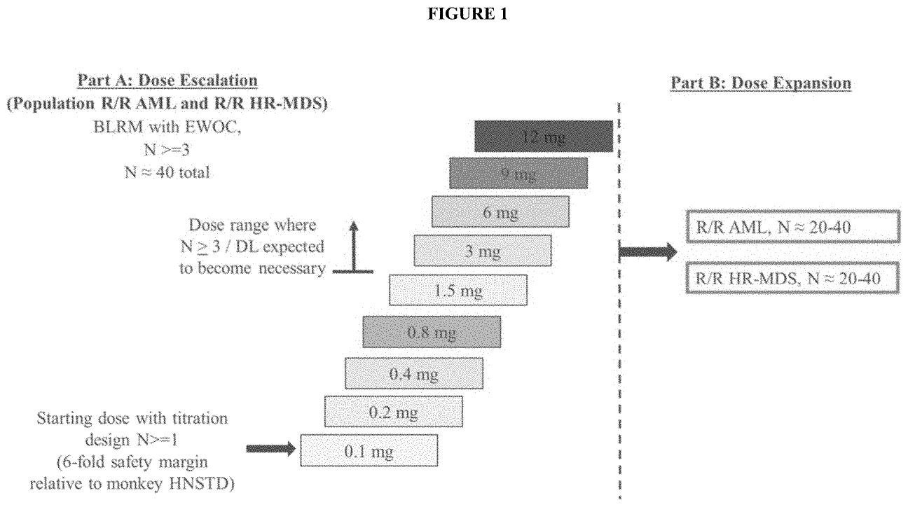

Dose and schedule findings will begin with evaluation of QD administration for 7 days followed by a 21-day recovery period (ie, Days 1-7 per 28-day cycle). Provisional dose escalation steps for QD Days 1-7 schedule are shown in FIG. 1 and includes dose increments no more than 100%. The SRC may elect to modify dose levels to reduce dose increments and will make the decisions to evaluate a higher dose cohort, additional subjects within a dose cohort, open an intermediate dose cohorts or alternative dosing schedules and/or declare an MTD after review of BLRM guidance results, and available safety, PK, PD and preliminary efficacy data when the required number of subjects per dose level has been observed for at least the DLT period.

Upon review of safety and/or available PK/PD, and preliminary efficacy analyses, daily administration may be adjusted to abbreviate dosing duration over 5 days (ie, Days 1-5 per 28-day cycle) or to extend the duration over 10 to 14 days (ie, Days 1-10 to Days 1-14 per 28-day cycle) with the dose tolerated in Days 1-7 schedule. In addition, a BIW dosing schedule (Days 1, 4, 8, 11, 15, 18, 22, 25 per 28-day cycle) may be evaluated. Finally, potential adjustments to BIW dosing may be made with dosing over 2 weeks (Days 1, 4, 8, 11 per 28-day cycle) or with dosing over 3 weeks (Days 1, 4, 8, 11, 15, 18 per 28-day cycle). If SRC determines that BIW dosing schedule should be evaluated based on the review of the data from the daily dosing schedule, a protocol amendment will be necessary to provide the rationale for BIW dosing and starting dose prior to enrolling subjects in BIW dosing cohort. The dose escalation decisions in all the schedules would be guided by the BLRM approach adjusted with each schedule.

In Part A, after the first dose is administered in any cohort, subjects in each cohort are observed for the DLT observation period of at least 28 days, and up to 42 days in subjects with Grade 4 neutropenia and/or thrombocytopenia, with a hypocellular bone marrow and no greater than 5% marrow blasts in the absence of underlying MDS, before the next higher, protocol specified dose cohort can begin.

In the dose escalation cohorts with more than one subject, the first subject's Cycle 1 Day 1 dose will be staggered by 1 day with the next subject's first dose unless an equivalent or higher Compound 1 dose has already been tested on an alternative schedule.

Individual dose levels may be backfilled with up to 9 subjects per dose cohort to confirm safety, PK, PD, and preliminary efficacy observations to enable selection of an MTD or preliminary RP2D for evaluation in Part B.

### BLRM-Based Dose Escalation

Observation of a DLT will mandate use of the BLRM for all subsequent dose escalation steps with initial treatment of a minimum of 3 to 6 subjects per dose level and observation for at least the DLT period before BLRM-based dose selection guidance for subsequent subjects is provided to the SRC for consideration. In addition, the posterior probability of DLT rate exceeding 25% will be estimated. The dose escalation is considered safe if the posterior probability is no more than 50%.

Informed by GLP toxicology studies and any available clinical safety and PK data, the BLRM will be adjusted to handle different schedules for QD and BIW administration while concurrent dose escalation in QD and BIW schedules will be allowed.

The BLRM permits alterations in the dose increments based on the observed DLTs; however, the maximum daily dose for the next cohort will not exceed a 100% increment from the prior dose. The MTD is the highest dose at which less than 33% of the population treated with Compound 1 experience a DLT in the first cycle and at least 6 evaluable subjects treated at this dose. Dose escalation within a dose administration schedule will proceed until the MTD or RP2D is established or the SRC decides to pursue dose finding in an alternate dose administration schedule.

The RP2D may be selected based on PD analyses before a safety limit is identified. If it is selected based on safety, the recommendation by BLRM will be the dose with the highest probability that the DLT rate will fall in the target toxicity and will always satisfy the EWOC principle that it should be unlikely (<30% posterior probability) that the DLT rate at the next dose will exceed 0.33. The ultimate selection of the MTD/RP2D by SRC will also be guided by the modeling and simulation to evaluate PK, PD and exposure-response to select a dose with both the highest probability of having a target toxicity <25% and lowest probability of underdosing for efficacy.

### Part B-Cohort Expansion

Following identification of an MTD or a preliminary RP2D in Part A, subjects may be enrolled onto Part B with approximately 20 to 40 subjects per cohort. The SRC will decide which doses and schedules to evaluate and may choose to expand more than one dose or schedule in Part B based on BLRM guidance results, review of safety, PK, PD and preliminary efficacy data from Part A.

The SRC will continue to review safety, PK, PD, and preliminary efficacy data regularly throughout the study and make recommendations about study continuation and dose and schedule modification, as appropriate. When approximately 20 subjects per cohort are deemed response evaluable based on meeting respective disease response criteria for complete remission, treatment failure or progression, or based on having completed the end-of-cycle response assessment with bone marrow (BM) examination after the second cycle of therapy, whichever occurs earlier, a futility analysis will be performed utilizing the Bayesian method.

The SRC may stop accrual to respective cohorts at any time and will decide based on review of safety, PK, PD, and preliminary efficacy data, including the futility analysis, whether continued accrual up to approximately 40 response evaluable subjects per cohort is warranted.

### Study Population

Subjects 18 years or older, with relapsed or refractory AML and relapsed or refractory higher risk MDS as defined by World Health Organization (WHO) criteria who have failed or are ineligible for all available therapies which may provide clinical benefit will be enrolled in Part A of the study. In Part B, subjects 18 years or older, with R/R AML and R/R MDS will be enrolled in 2 separate cohorts. Both subject populations must not have known tumor protein 53 (TP53) mutation or loss of heterozygosity for TP53 which is determined locally as a standard of care pre-study evaluations and fulfill additional criteria for high unmet medical need specified in eligibility criteria.

### Length of Study

Enrollment is expected to take approximately 42 to 48 months to complete (˜24 to 30 months for dose escalation and schedule development, and ˜18 months for expansion). Completion of active treatment and post-treatment follow-up is expected to take an additional 6 to 24 months. The entire study is expected to last up to approximately 4 to 6 years.

### Study Treatments

Compound 1 will be supplied in capsules of appropriate strengths (including but not limited to 0.1 mg, 0.5 mg and 2 mg strength) by the Sponsor and labeled appropriately as investigational product (IP) for this study. Compound 1 will be supplied according to local clinical study agreement and in accordance with local guidelines.

Compound 1 will be administered orally QD or BIW according to the assigned treatment schedule as outlined in Table below, every 28 days for 6 cycles.

| | | Compound 1 |
|---|---|---|
| | | Administration |
| Schedule Type | Schedule Name | (per 28-days cycle) |
| Daily (initial) | QD × 7 | Day 1-7 |

Alternate options

| | | |
|---|---|---|
| Daily | QD × 5 | Day 1-5 |
| | QD × 10 | Day 1-10 |
| | QD × 14 | Day 1-14 |
| Twice a week | BIW × 4 wks | Day 1, 4, 8, 11, |
| (implemented | | 15, 18, 22, 25 |
| after protocol | BIW × 3 wks | Day 1, 4, 8, 11, 15, 18 |
| amendment) | BIW × 2 wks | Day 1, 4, 8, 11 |

Those who demonstrate benefit from treatment without unacceptable toxicity (complete remission [CR], partial remission [PR], or stable disease with demonstrated clinical benefit eg, reduction in transfusion burden) may continue treatment beyond Cycle 6 up to 2 years, after discussion with Medical Monitor, until loss of that benefit, unacceptable toxicity, or subject/physician decision to withdraw. Subjects may continue to receive Compound 1 beyond 2 years at the discretion of the Investigator and in consultation with the Sponsor's Medical Monitor. Dosing may be evaluated and adjusted per dose escalation rules depending on safety, PK, PD and preliminary efficacy analyses.

### Overview of Key Efficacy Assessments

The primary efficacy variable is leukemia response rate.

All treated subjects will be included in the efficacy analyses. Leukemia response will be determined by the Investigator. Assessment will be based on the International Working Group Response Criteria in AML (Cheson, J Clin Oncol 2003;21(24):4642-9).

A descriptive analysis of evidence of antileukemic activity will be provided based on clinical, laboratory, molecular, and cytogenetic assessments by Investigator, which includes assessment of bone marrow blast percentage, bone marrow cytogenetics, molecular genetic studies to evaluate molecular responses, bone marrow flow cytometry, platelet count, and absolute neutrophil count.

Response criteria will be summarized by best overall response categories: complete remission rate (CRR), and objective response rate (ORR). The ORR includes all responses of complete remission (CR) (ie, morphologic leukemia-free state, morphologic CR, cytogenetic CR, molecular CR, and morphologic CR with incomplete blood recovery), and partial remission.

The efficacy variable of focus will be ORR and CRR. Other measures of clinical activity including overall survival (OS), relapse free survival (RFS), progression-free survival (PFS), event-free survival, duration of remission, duration of response, and time to remission/response will be summarized.

### Overview of Key Safety Assessments

Disease assessments by BM aspiration and biopsy, along with complete blood counts and examination of peripheral blood smears will be performed.

In addition, BM aspirates and biopsies must be collected in order to confirm complete remission (CR) or morphologic CR with incomplete hematologic recovery (CRi) and morphologic CR with partial hematologic recovery (CRh), relapse after CR, CRi, or CRh (as assessed by the Investigator based on complete blood counts with white blood cell differential results), or progressive disease. Additional aspirates and/or biopsies will be collected as clinically indicated (eg, Cycle 1 Day 15, based on Investigator's medical assessment).

Bone marrow aspirates and biopsies are to be evaluated for morphology, flow cytometry, karyotype, and molecular studies.

Efficacy analyses for acute myeloid leukemia (AML) will use European LeukemiaNet (ELN) criteria (Döhner et al., Diagnosis and management of AML in adults: 2017 ELN recommendations from an international expert panel, Blood 2017;129(4):424-447) which incorporate and extend the International Working Group (IWG) Response Criteria for AML (Cheson et al. Revised recommendations of the International Working Group for diagnosis, standardization of response criteria, treatment outcomes, and reporting standards for therapeutic trials in acute myeloid leukemia, J Clin Oncol 2003;21(24):4642-9). In addition, CRh will be reported for AML (Bloomfield et al. Time to repeal and replace response criteria for acute myeloid leukemia? Blood Rev. 2018:32(5)416-425) to further characterize treatment effects. Myelodysplastic syndromes (MDS) efficacy analyses will use IWG Response Criteria for Myelodysplasia (Cheson et al. Clinical application and proposal for modification of the International Working Group (IWG) response criteria in myelodysplasia, Blood 2006;108(2):419-25).

The efficacy variables for AML are: complete remission rate (CRR) defined as CR plus CRh plus CRi, minimal residual disease (MRD) negative CRR (CRR_{MRD-}), combined complete response rate (cCRR=CR+CRR_{MRD-}+CRi+CRh), morphologic Leukemia-free State (MLFS) rate (MLFSR), partial remission rate (PRR), stable disease rate (SDR), Progression Free Survival (PFS) rate at 3 and 9 months, Overall Survival (OS) rate at 6 and 12 months, and overall response rate (ORR). The ORR includes all responses of complete remission (CR) (ie, CRS-, morphologic CR, cytogenetic CR, molecular CR, and morphologic CR with incomplete blood recovery or CR with partial hematologic recovery), MLFS, and PR.

The efficacy variables for MDS will be CRR, marrow CR with hematologic improvement (HI) rate (mCRHIR), PR rate (PRR), hematologic improvement rate (HIR), SDR, PFS rate at 3 and 9 months, OS rate at 6 and 12 months and overall response rate (ORR=CR+mCR with HI+PR+HI) according to IWG Response Criteria for Myelodysplasia (Cheson et al. Clinical application and proposal for modification of the International Working Group (IWG) response criteria in myelodysplasia, Blood 2006;108(2):419-25) for MDS.

Other measures of clinical activity including OS, PFS, relapse-free survival (RFS), event-free survival (EFS), duration of remission, duration of response, time to transformation to AML (HR-MDS subjects only) and time to remission/response will be analyzed.

Subjects who have discontinued study treatment due to relapse or progression of disease or start of a new anticancer therapy will be followed for a minimum of 2 years, until they have died, are lost to follow up, withdraw consent for further data collection, or until study closure.

### Overview of Key Safety Assessments

The safety variables for this study include AEs (to be graded in severity according to the guidelines outlined in the National Cancer Institute [NCI] Common Terminology Criteria for Adverse Events [CTCAE] version 5.0, except for tumor lysis syndrome (TLS), which will be graded according to the Cairo-Bishop TLS grading system (Cairo et al. Tumour lysis syndrome: new therapeutic strategies and classification. Br J Haematol 2004;127:3-11), clinical safety laboratory variables, physical examinations, vital signs, exposure to study treatment, assessment of concomitant medications, Eastern Cooperative Oncology Group Performance Status (ECOG PS), electrocardiograms (ECGs) and pregnancy testing for subjects of childbearing potential.

### Overview of Key Pharmacokinetic Assessments

Blood samples will be collected at specified times for measurement of Compound 1 and Compound 2 in plasma. Urine will be collected in approximately 10 evaluable subjects in

Part A and/or Part B. All subjects in Part A will be required to participate in both intensive and sparse PK sampling schedules. Pharmacokinetic sampling is performed.

PK parameters (Cₘₐₓ, AUC₀₋₂₄, t½, CL/F, Vₛₛ/F, Tₘₐₓ as appropriate) of Compound 1 (and Compound 2 if data allows) will be calculated using noncompartmental analysis method based on the plasma concentration-time data. The Sponsor may conduct additional PK analyses in order to follow-up the safety of the study treatment or to better understand the progression of the disease or the disease's response to the study treatment.

### Overview of Pharmacodynamic Assessments

Pharmacodynamic biomarker samples will be collected before and during treatment from peripheral blood and a portion of BM tissue (aspirate and biopsy) to assess important PD endpoints such as the depth and duration of CK1α degradation, p53 stabilization, p21 induction, MIC-1 induction and composition of normal and transformed cells. Gene expression analyses of BM (or blood samples in case of dry tap) at screening and on treatment will also be carried out to further explore mechanisms of response and resistance retrospectively and to help guide future subject selection strategies. Additional PD markers may be identified from ongoing proteomics and gene expression and mutation profiling experiments of Compound 1 in AML cells and be incorporated into the first in human (FIH) study.

Reflecting the molecular diversity of AML, different MRD platforms are available for detecting MRD. As per ELN recommendation, MRD assessments will be performed in BM aspirates using either or both, multiparameter flow cytometry and molecular MRD by next generation sequencing.

The Sponsor may conduct additional analyses on the PD samples to follow-up on the safety of the study treatment or to better understand the progression of disease or responses to the study treatment.

### Statistical Methods

An accelerated titration design will be utilized until the occurrence of a DLT. Afterward, the two-parameter BLRM with EWOC together with the posterior probability of DLT rate exceeding 25% will be utilized to help guide Compound 1 dose escalation decisions and estimate the MTD during the escalation phase of the study with the final decisions being made by the SRC.

Statistical analyses will be performed by dose level and schedule (Part A) and tumor cohort (Part B) as needed or applicable. Subjects treated at the RP2D and schedule in dose expansion (Part B) will be combined with subjects treated at the comparable dose and schedule in dose escalation (Part A) for safety analyses. All analyses will be descriptive in nature. Summaries of subject disposition, demographic and baseline disease characteristics, treatment exposure, efficacy, safety, PK, and PD will be provided. Categorical data will be summarized by frequency distributions (number and percentages of subjects) and continuous data will be summarized using descriptive statistics (means, standard deviations, medians, minimums, and maximums).

Efficacy analysis will be based on the Treated/Safety Population and repeated for Efficacy Evaluable (EE) Population for primary efficacy endpoints with the result using the Treated Population considered primary. The primary efficacy variables of focus will be ORR and CRR. Efficacy outcomes will be summarized using frequency tabulations for CRR/ORR and PFS/OS rate at selective timepoints, the point estimates and 95% CIs of CRR/ORR and PFS/OS rate will be reported as well. Additional efficacy variables including OS, RFS, PFS, EFS, duration of remission, duration of response, time to transformation to AML (HR-MDS subjects only) and time to remission/response will be analyzed.

Severity of treatment-emergent AEs (TEAEs) will be summarized by NCI-CTCAE v. 5.0, except for tumor lysis syndrome (TLS), which will be graded according to the Cairo-Bishop TLS grading system (Cairo et al. Tumour lysis syndrome: new therapeutic strategies and classification. Br J Haematol 2004;127:3-11). The frequency of TEAEs will be tabulated by Medical Dictionary for Regulatory Activities (MedDRA) Version 20.1 or higher system organ class and preferred term. Grade 3 or 4 TEAEs, TEAEs leading to discontinuation of Compound 1, study drug-related TEAEs, deaths, and serious AEs (SAES) will be tabulated separately. Changes from baseline in selected laboratory analytes, vital signs, 12-lead ECGs will be summarized. All data will also be presented in by-subject listings. All summaries of safety data will be conducted using subjects receiving any Compound 1 (the Treated Population).

All biomarker-related data presentations will be based on treated subjects with at least one baseline and one biomarker assessment, unless specified otherwise. Descriptive statistics will be presented for baseline, post-baseline values, and changes from baseline or percent change from baseline of each biomarker.

The summary statistics will also be provided for Compound 1 PK parameters by study day and dose cohort and be presented in a tabular form based on treated subjects with at least one measurable concentration of Compound 1.

Exploration of PK, PD, safety and efficacy relationships may be assessed.

During the Part A dose escalation, up to approximately 40 subjects will be enrolled. The actual number depends on the number of schedules/dose levels required to determine the MTD or RP2D.

During the Part B dose expansion approximately 20-40 subjects per R/R AML cohort and approximately 20-40 subjects per R/R HR-MDS cohort may be enrolled. When approximately 20 subjects per cohort are deemed response evaluable based on meeting criteria for complete remission, treatment failure or progression according ELN (Doehner et al. Blood 2017 Jan. 26;129(4):424-447) or IWG criteria (Cheson et al. Blood 2006;108(2):419-25) for AML and MDS, respectively, or based on having completed the end-of-cycle response assessment with bone marrow examination after the second cycle of therapy, whichever occurs earlier, a Bayesian method will be utilized for futility analysis. The following stopping criterions based on the posterior distributions of the combined complete response rates for R/R AML (combined rate of CR+CRM_{MRD-}+CRi+CRh=cCR) and R/R HR-MDS (CRR) will be calculated:
R/R AML: Probability of (cCRR>0.25|data, a, b)≤0.1
R/R HR-MDS: Probability of (CRR>0.20|data, a, b)≤0.2
   Where a, b are parameters of Beta distribution. The weakly informative priors Beta (0.25, 0.75) and Beta (0.20, 0.80) are considered in estimation of futility boundary for R/R AML and R/R HR-MDS, respectively.
   For R/R AML a cCRR≤15% and for R/R HR-MDS a CRR≤10% will be considered non-interesting. A CRR of ≥25% in R/R AML and ≥20% in R/R HR-MDS will permit further accrual.

The enrollment to the respective cohort may stop for futility when the stop criterion above is met (≤2 responders out of 20 subjects for R/R AML or R/R HR-MDS), which implies that there is a 10% or less probability that the cCRR is higher than 25% in R/R AML or 20% or less probability that the CRR is higher than 20% in R/R MDS. Otherwise, enrollment may continue until up to approximately 40 efficacy evaluable subjects per cohort.

The SRC may stop accrual to cohorts at any time and will make the final decision on whether accrual of additional subjects is warranted based on review of the futility analysis as well as of safety, PK, PD and other preliminary efficacy analyses.

With the sample size of up to 40 subjects per cohort and assuming an observed cCRR of 25% in R/R AML and 20% CRR in R/R HR-MDS, the two-sided 90% Wilson score confidence interval will be 16% to 38% for R/R AML and 12% to 32% for R/R HR-MDS, which would mean the true cCRR and CRR, respectively, will exclude non-interesting efficacy signals with 90% certainty.

### Inclusion Criteria

Subjects must satisfy the criteria below to be enrolled in the Dose Escalation (Part A) or the Dose Expansion (Part B) of this study.
1. Subject is ≥18 years of age, at the time of signing the ICF.
2. Subject must understand and voluntarily sign an ICF prior to any study-related assessments/procedures being conducted.
3. Subject is willing and able to adhere to the study visit schedule and other protocol requirements.
4. Relapsed or refractory AML and R/R HR-MDS as defined by the WHO criteria who have failed or are ineligible for all available therapies which may provide clinical benefit, and have no known TP53 mutation or loss of heterozygosity for TP53 or chromosome p17 as determined by NGS, polymerase chain reaction, fluorescence in situ hybridization (FISH), or karyotype analysis obtained during standard of care pre-study evaluations (testing for TP53 mutation is not mandatory prior to study entry).
5. Subject has Eastern Cooperative Oncology Group Performance Status of 0 to 2.
6. Subjects must have the following screening laboratory values:
   Total White Blood Cell count (WBC) <25×10⁹/L prior to first infusion. Treatment with hydroxyurea to achieve this level is allowed, but hydroxyurea should be discontinued prior to Day 1.
   Potassium, calcium and magnesium within normal limits or correctable with supplements (calcium corrected for albumin or ionized calcium can be assessed for eligibility if total calcium is low).
   Aspartate aminotransferase (AST)/serum glutamic oxaloacetic transaminase (SGOT) and alanine aminotransferase (ALT)/serum glutamic pyruvic transaminase (SGPT) ≤3.0×upper limit of normal (ULN), unless considered due to leukemic organ involvement, in which case AST and ALT can be ≤5.0×ULN.
   Uric acid≤7.5 mg/dL (446 μmol/L). Prior and/or concurrent treatment with hypouricemic agents (eg, allopurinol, rasburicase) are allowed.
   Serum total bilirubin≤1.5×ULN, unless considered due to Gilbert's syndrome (eg, a gene mutation in UGT1A1). If Gilbert's syndrome, total bilirubin must be <3×ULN.
   Estimated serum creatinine clearance of ≥60 mL/min using the Cockcroft-Gault equation. Measured creatinine clearance from a 24-hour urine collection is acceptable if clinically indicated.
   INR<1.5×ULN and PTT<1.5×ULN.
7. Per the pregnancy prevention plan (PPP):
   A subject of childbearing potential (SCBP) must undergo pregnancy testing on site and have 2 negative pregnancy tests as verified by the Investigator prior to starting Compound 1. The subject must agree to ongoing pregnancy testing during the course of the study, and after the end of study treatment based on the frequency outlined in the PPP. This applies even if the subject practices true abstinence from heterosexual contact. The subject may not receive Compound 1 until the Investigator has verified that the result of the pregnancy test is negative.
      a negative serum pregnancy test (sensitivity of at least 25 mIU/mL) at Screening
      a negative serum or urine pregnancy test (Investigator's discretion) within 24 hours prior to Cycle 1 Day 1 of study treatment (note that the Screening serum pregnancy test can be used as the test prior to Day 1 study treatment if it is performed within the prior 24 hours). A serum or urine pregnancy test (investigators discretion) must also be performed at the end of study for each FCBP.
         Note: A subject of childbearing potential is a sexually mature individual who 1) has achieved menarche at some point, 2) has not undergone a hysterectomy (the surgical removal of the uterus) or bilateral oophorectomy (the surgical removal of both ovaries) or 3) has not been naturally postmenopausal (amenorrhea following cancer therapy does not rule out childbearing potential) for at least 24 consecutive months (ie, has had menses at any time during the preceding 24 consecutive months).
         True abstinence is acceptable when this is in line with the preferred and usual lifestyle of the subject. Periodic abstinence (eg, calendar, ovulation, symptothermal, post-ovulation methods) and withdrawal are not acceptable methods of contraception.
   Unless practicing complete abstinence from heterosexual intercourse, sexually active subject of childbearing potential must agree to use adequate contraceptive methods as specified in PPP.
      Subject must agree to use 2 reliable forms of contraception simultaneously, without interruption, for at least 28 days before starting Compound 1, throughout the entire duration of Compound 1 treatment, during dose interruptions, and for at least 28 days after the last dose of Compound 1.
   Unless practicing complete abstinence from heterosexual intercourse, sexually active subjects who are able to impregnate their partner (including those who have had a vasectomy) must use barrier contraception (condoms) when engaging in sexual activity with a partner with childbearing potential as specified in PPP.
      Complete abstinence is only acceptable in cases where this is the preferred and usual lifestyle of the subject.
      Subjects who are able to impregnate their partner must agree to use a condom during sexual contact with a pregnant partner or a partner with childbearing potential while taking Compound 1, during dose interruptions, and for at least 92 days after the last dose of Compound 1, as specified in PPP.
   Subjects must agree to abstain from breastfeeding or providing breast milk for the duration specified in the PPP.
   Subjects of chiuldberaing potential must avoid conceiving for 28 days after the last dose of Compound 1.
   Subjects who are able to impregnate their partrner must agree not to donate semen or sperm while receiving Compound 1, during dose interruptions, or for at least 92 days following the last dose of Compound 1, as specified in the PPP.
   All subjects must:
      Understand that Compound 1 could have a potential teratogenic risk.
      Agree to abstain from donating blood for the duration specified in the PPP.
      Be counseled about pregnancy precautions and risks of fetal exposure.

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed:

1. A method for treating, preventing, managing, or ameliorating a disease selected from acute myeloid leukemia and myelodysplastic syndrome comprising administering to a subject in need thereof N-((R)-1-(3-chloropyridin-2-yl)-2,2,2-trifluoroethyl)-2-((S)-2,6-dioxopiperidin-3-yl)-1-oxoisoindoline-5-carboxamide, which has the following structure: 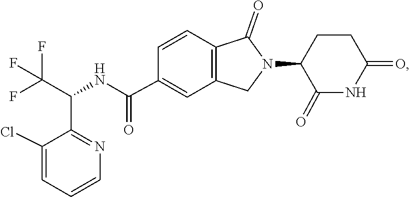 [SMILES: O=C1CC[C@H](N2Cc3cc(C(=O)N[C@H](c4ncccc4Cl)C(F)(F)F)ccc3C2=O)C(=O)N1]
or a stereoisomer or mixture of stereoisomers, isotopologue, pharmaceutically acceptable salt, tautomer, solvate, hydrate, co-crystal, clathrate, or polymorph thereof (Compound 1), wherein Compound 1 is administered to the subject in a dose of about 0.1 mg to about 20 mg.

2. The method of claim 1, wherein the disease is acute myeloid leukemia.

3. The method of claim 2, wherein the acute myeloid leukemia is refractory or relapsed acute myeloid leukemia.

4. The method of claim 1, wherein the disease is myelodysplastic syndrome.

5. The method of claim 4, wherein the myelodysplastic syndrome is refractory or relapsed myelodysplastic syndrome.

6. The method of claim 4, wherein the myelodysplastic syndrome is refractory or relapsed high risk myelodysplastic syndrome.

7. The method of claim 1, wherein Compound 1 is administered on days 1 to 5 of a 28 day treatment cycle.

8. The method of claim 1, wherein Compound 1 is administered on days 1 to 7 of a 28 day treatment cycle.

9. The method of claim 1, wherein Compound 1 is administered on days 1 to 10 of a 28 day treatment cycle.

10. The method of claim 1, wherein Compound 1 is administered on days 1 to 14 of a 28 day treatment cycle.

11. The method of claim 1, wherein Compound 1 is administered on days 1, 4, 8, 11, 15, 18, 22 and 28 of a 28 day treatment cycle.

12. The method of claim 1, wherein Compound 1 is administered on days 1, 4, 8, 11, 15 and 18 of a 28 day treatment cycle.

13. The method of claim 1, wherein Compound 1 is administered on days 1, 4, 8 and 11 of a 28 day treatment cycle.

14. The method of claim 7, wherein the treatment cycle is repeated at least once.

15. The method of claim 7, wherein the treatment cycle is repeated 2 to 4 times.

16. The method of claim 1, wherein Compound 1 is administered in a dose of about 0.1 mg/day, 0.2 mg/day, 0.4 mg/day, 0.5 mg/day, 0.8 mg/day, 1 mg/day, 1.5 mg/day, 2 mg/day, 3 mg/day, 6 mg/day, 9 mg/day and 12 mg/day.

17. The method of claim 1, further comprising administering a therapeutically effective amount of a second active agent or a supportive care therapy.

18. The method of claim 1, wherein the subject is a patient 18 years or older.

19. The method of claim 1, wherein the subject does not have a TP53 mutation.
